(19) United States
(12) Patent Application Publication
(10) Pub. No.: US 2016/0039897 A1
(43) Pub. Date: Feb. 11, 2016

(54) COMPOUNDS AND METHODS FOR TREATING OBESITY AND CONTROLLING WEIGHT

(71) Applicant: THE UNIVERSITY OF NORTH CAROLINA AT CHAPEL HILL, Chapel Hill, NC (US)

(72) Inventor: David Clemmons, Chapel Hill, NC (US)

(21) Appl. No.: 14/775,103

(22) PCT Filed: Mar. 12, 2014
(86) PCT No.: PCT/US2014/024531

§ 371 Date: Sep. 11, 2015

Related U.S. Application Data
(60) Provisional application No. 61/777,773, filed on Mar. 12, 2013.
(60) Provisional application No. 61/876,592, filed on Sep. 11, 2013.

(51) Int. Cl. C07K 14/47; A61K 38/17; A61K 45/06

(52) U.S. Cl. CPC C07K 14/4743; A61K 45/06; A61K 38/1754; A61K 38/00

(57) ABSTRACT

The present invention provides peptides and peptide compositions and methods of their controlling body weight and treating obesity.

### STATEMENT OF PRIORITY

This application claims the benefit, under 35 U.S.C. §371 national phase application of International application Serial No. PCT/US2014/024531, filed Mar. 12, 2014, which claims the benefit, under 35 U.S.C. §119(e), of U.S. Application Ser. No. 61/777,773, filed Mar. 12, 2013 and U.S. Application Ser. No. 61/876,592, filed Sep. 11, 2013, the entire contents of each of which are incorporated by reference herein.

### STATEMENT OF GOVERNMENT SUPPORT

This invention was made with government support under Grant Nos. AG02331 and AR061164 awarded by the National Institutes of Health. The government has certain rights in this invention.

### FIELD OF THE INVENTION

The present invention concerns peptides, pharmaceutical formulations containing the same, and methods of use thereof in controlling weight and treating obesity.

### BACKGROUND OF THE INVENTION

Insulin-like growth factor binding protein-2 (IGFBP-2) is a 36,000 dalton protein that is a member of the IGFBP family. There are six forms of high affinity IGF binding proteins. In addition to binding the insulin-like growth factors I and II and acting as transport proteins, these proteins have been shown to have some actions that are independent of their ability to bind to IGFs.

IGFBP-2 is the second most abundant binding protein in serum. It circulates in concentrations in humans that vary between 100-600 ng/ml. Protein concentrations are high during fetal life and at birth and fall progressively during childhood and adolescence. There is a slight rise, an approximately 25% increase that occurs between 60-80 years. Serum concentrations of IGFBP-2 are regulated by hormones and nutrients. Fasting causes a significant increase in IGFBP-2 and feeding (particularly feeding protein) restores concentrations to normal. Concentrations are also suppressed by administration of insulin or growth hormone, and are increased by insulin-like growth factor-I (IGF-1). This may be due in part to suppression of growth hormone and insulin, both of which are suppressed by administering IGF-1.

The present invention overcomes previous shortcomings in the art by providing peptides of IFGBP-2 and methods of their use in treating obesity and controlling weight.

### SUMMARY OF THE INVENTION

In one aspect, the present invention provides an isolated peptide comprising the amino acid sequence: X₁ X₂ X₃ X₄ X₅ X₆ X₇ X₈ X₉ X₁₀ X₁₁ X₁₂ X₁₃ X₁₄ X₁₅ X₁₆ X₁₇ X₁₈, wherein:
X₁ is K, H or R;
X₂ is H, R or K;
X₃ is G, A or P;
X₄ is L, R, I or V;
X₅ is Y, F or M;
X₆ is N or Q;
X₇ is L, V or I;
X₈ is K, R or H;
X₉ is Q, N or S;
X₁₀ is C;
X₁₁ is K, H or R;
X₁₂ is M, F, W, or Y;
X₁₃ is S, T, N or Q;
X₁₄ is L, V or I;
X₁₅ is N, Q or S;
X₁₆ is G, A, S or P;
X₁₇ is Q, N, S or T; and
X₁₈ is R, K or H.

In a further aspect, the present invention provides an isolated peptide comprising the amino acid sequence: X₁ X₂ X₃ X₄ X₅ X₆ X₇ X₈ X₉ X₁₀ X₁₁ X₁₂ X₁₃, wherein:
X₁ is K, H or R;
X₂ is H, R or K;
X₃ is G, A or P;
X₄ is L, R, I or V;
X₅ is Y, F or M;
X₆ is N or Q;
X₇ is L, V or I;
X₈ is K, R or H;
X₉ is Q, N or S;
X₁₀ is C;
X₁₁ is K, H or R;
X₁₂ is M, F, W, or Y; and
X₁₃ is S, T, N or Q.

In one aspect, the present invention provides: a) a peptide comprising the amino acid sequence KHGLYNLKQCKMSLNGQR; b) a peptide comprising the amino acid sequence KHGLYNLKQCKMSLNGQR, wherein the K at position 1 is substituted with R or H, the H at position 2 is substituted with R or K, the K at position 8 is substituted with R or H, the K at position 11 is substituted with R or H, the R at position 18 is substituted with K or H, in any combination; or c) a pharmaceutically acceptable salt of any of (a) or (b) above, wherein the peptide is not a full length insulin-like growth factor binding protein (IGFBP-2).

In a further aspect, the peptide of this invention can comprise a polyalkylene glycol moiety coupled to the N terminus thereof, the C terminus thereof, or both the N terminus and C terminus thereof (e.g., via a reactive cysteine residue included at the N terminus and/or C terminus of the peptide).

In an additional aspect, the peptide of this invention can be present in a composition comprising a pharmaceutically acceptable carrier. In some aspects, the composition can further comprise a weight control agent, which can be, but is not limited to, an appetite suppressant, a lipase inhibitor, an antidepressant, an anti-seizure agent, anti preadipocyte differentiation factor and any combination thereof.

In additional embodiments, the present invention provides a method of inhibiting fat cell differentiation in a subject, comprising administering to the subject an effective amount of a peptide or composition of this invention.

Also provided herein is a method of inhibiting weight gain in a subject, comprising administering to the subject an effective amount of a peptide or composition of this invention.

In some embodiments, the present invention provides a method reducing weight in a subject, comprising administering to the subject an effective amount of a peptide or composition of this invention.

Furthermore, the present invention provides a method of reducing fat mass in a subject, comprising administering to the subject an effective amount of a peptide or composition of this invention.

In a further aspect, the present invention provides a method for controlling body weight in a subject (e.g., a subject in need thereof), comprising administering to said subject the composition or peptide of this invention in an amount effective to control body weight.

Additionally, the present invention provides a method of treating obesity in a subject (e.g., a subject in need thereof), comprising administering to said subject the composition or peptide of this invention in an amount effective to treat obesity.

A further aspect of the invention is the use of a peptide or composition as described herein for carrying out a method as described herein, and/or for the preparation of a medicament for carrying out a method as described herein.

The present invention is explained in greater detail below.

### BRIEF DESCRIPTION OF THE DRAWINGS

FIGS. 1A-B. IGFBP-2 and its heparin-binding domains inhibit the differentiation of preadipocytes isolated from IGFBP-2 null mice. (A) Primary preadipocytes from IGFBP-2^{-/-} mice were isolated from inguinal fat pads following the procedure described herein. Two days after reaching confluence, cells were treated with differentiation medium (DM, lanes 1-8). This medium was then supplemented with HBD1 peptide (HBD1, lane 2); scrambled HBD1 peptide (HBD1 Ctrl, lane 3); HBD2 peptide (HBD2, lane 4); IGFBP-5 C-terminal HBD peptide (HBD2 Ctrl, lane 5); IGFBP-2 protein with mutated HBD1 sequence (HBD1 mutant IGFBP2, lane 6); IGFBP-2 with mutated HBD2 sequence (HBD2 mutant IGFBP2, lane 7); or native IGFBP-2 (WT IGFBP-2, lane 8). The cell lysates were immunoblotted (IB) with anti-Adiponectin, aP2, and PPARγ antibodies, respectively. As a loading control, the blots were immunoblotted with an anti-β-actin antibody. Quantitative analysis of the results from 3 separate experiments was performed and the results are expressed in relation to β-actin expression. Each value represents mean±SE. * p<0.05 and **p<0.01 denote a significant difference between two treatments. P, NS indicates no significant difference between two treatments. (B) Preadipocytes isolated from IGFBP-2^{-/-} mice were treated with either differentiation medium (DM), DM plus wild type IGFBP-2 (Wt IGFBP2) or DM plus IGF-I non binding IGFBP2 (IGF-I NB IGFBP-2). The cell lysates were immunoblotted (IB) with anti-Adiponectin, aP2, and. PPARγ antibodies, respectively. As a loading control, the blots were immunoblotted with an anti-β-actin antibody.

FIG. 2. Oil red O staining of IGFBP-2^{-/-} preadipocytes exposed to IGFBP-2, IGFBP-2 mutants or peptides containing the different heparin binding domains. Primary preadipocytes from IGFBP-2^{-/-} mice were cultured in the standard medium. Two days after they reached confluence, cells were treated with either differentiation medium (DM) alone or with this medium plus a peptide containing IGFBP-2 HBD1 sequence (HBD1), a peptide containing the scrambled HBD1 sequence (HBD1 Ctrl), a mutant form of IGFBP-2 containing a substituted HBD1 sequence (HBD1 MP), wild type bovine IGFBP-2 (WT IGFBP-2), a peptide containing IGFBP-2 HBD2 sequence (HBD2), a peptide containing the IGFBP-5 C-terminal HBD sequence (HBD2 Ctrl), or a mutant form of IGFBP-2 containing a substituted HBD2 sequence (HBD2 MP). After 48 hrs, the media were changed to standard medium plus 0.5 mM insulin. The media were changed to standard medium after additional 48 hrs. The cultures were stained with Oil Red O following the procedure herein. The results were quantified using ImageJ software and expressed as Oil Red O positive area (pixels) divided by whole area (pixels). ***p<0.001 indicates the significant differences between two treatments. P, NS, indicates no significant difference between two treatments. A representative image of 3 independent experiments is shown.

FIGS. 3A-C. The heparin binding domains of IGFBP-2 suppress weight gain in IGFBP-2^{-/-} mice but do not affect glucose metabolism and food intake. IGBPP-2^{-/-} mice were treated with either a pegylated synthetic peptide containing IGFBP-2 HBD1sequence (PegHBD1, n=8), the IGFBP-2 HBD2 sequence (PegHBD2, n=10) or a pegylated synthetic control peptide (PegCtrl, n=18) following the procedures described herein for 12 weeks. Wild type mice (Wt, n=8) were injected with phosphate buffered saline. (A) Average body weight gain of mice at weekly time points was calculated for each group. (B) Average daily feed intake was calculated for each group. (C) Oral glucose tolerance tests were performed in all groups of mice after 12 weeks of treatment following a procedure described herein. Each bar value represents mean±SE. Different letters represent significant differences between two treatments.

FIGS. 4A-B. The heparin binding domains of IGFBP-2 suppress body fat mass gain and prevent the loss of body lean mass in IGFBP-2^{-/-} mice. IGFBP-2^{-/-} mice were treated as described in the legend to FIG. 3. Body fat and lean mass from each group were analyzed using Echo-MRI scanning at weeks 0 and 12, respectively. The changes in total fat mass or lean mass between weeks 0 and 12 in each group are presented as the changes in fat or lean mass expressed as a percentage of body weight (A,B). Each bar value represents mean±SE. *p<0.05 indicates a significant difference between two treatments.

FIGS. 5A-C. The heparin binding domains of IGFBP-2 suppress inguinal fat and visceral fat development in IGFBP-2^{-/-} mice. IGFBP-2^{-/-} mice were treated as described in the description of FIG. 3. At sacrifice, the liver and heart of each mouse were collected and weighed (A). The inguinal fat and visceral fat were dissected following a procedure described herein and weighed. The results are shown as the inguinal fat or visceral fat mass expressed as a percentage of body weight (B,C). Each bar value represents mean SE. *p<0.05 and **p<0.01 indicate the significant differences between two treatments. P, NS, indicates no significant difference between two treatments.

FIGS. 6A-C. The heparin binding domains of IGFBP-2 decrease fat pad triglyceride content and serum adiponectin levels whereas HBD2 stimulates serum leptin in IGFBP-2^{-/-}mice. IGFBP-2^{-/-} mice were treated as described in the description of FIG. 3. At the end of week 12, blood was collected from each mouse before they were sacrificed. (A) Triglyceride levels in the right inguinal fat pad were measured following the procedure described herein. Serum adiponectin (B) and leptin levels (C) were measured following manufacturer's instructions. Each bar value was expressed as mean±SE. *p<0.05 and ** p<0.01 indicate significant differences between two treatments.

FIG. 7. Primary preadipocytes from IGFBP-2^{-/-} mice were cultured in the standard medium. (A) Two days after they reached confluence, cells were treated with either with differentiation medium (DM) alone or this medium plus with wild type IGFBP-2 (Wt IGFBP2), a peptide containing mouse IGFBP-2 HBD1 sequence (mHBD1), and a peptide containing human IGFBP-2 HBD1 sequence (hHBD1). The cell lysates were immunoblotted (IB) with an anti-Adiponectin antibody. As a loading control, the blot was immunoblotted with an anti-β-actin antibody. (B) Cells were treated with either differentiation medium (DM) alone or this medium plus with a HBD1 peptide (HBD1), a HBD2 peptide (HBD2), IGF-I (100 ng/ml, DM+IGF-1), IGF-I plus HBD1 (DM+I+HBD1), IGF-I plus HBD2 (DM+I+HBD2) and wild type IGFBP-2 (Wt IGFBP2). The cell lysates were immunoblotted (IB) with anti-Adiponectin, aP2, and PPARγ antibodies, respectively. As a loading control, the blots were immunoblotted with an anti-β-actin antibody.

FIG. 8. After four weeks of HBD-2 treatment of ovariectomized (OVX) mice, body weight and total fat mass are significantly decreased.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all patents, patent publications and non-patent documents cited herein are incorporated herein by reference in their entirety.

The present invention is based on the unexpected discovery of a peptide contained within the IGBP-2 protein that inhibits fat cell differentiation. Such a peptide has therapeutic use in controlling body weight and treating obesity. The present invention is further based on the discovery that peptides of IGFBP-2, (e.g., the heparin binding domain 2 (HBD2)) can be employed in methods of treating obesity, methods of weight control and methods of inhibiting fat development.

Thus, in certain embodiments, the present invention provides an isolated peptide comprising the amino acid sequence: X₁ X₂ X₃ X₄ X₅ X₆ X₇ X₈ X₉ X₁₀ X₁₁ X₁₂ X₁₃ X₁₄ X₁₅ X₁₆ X₁₇ X₁₈, wherein:
X₁ is K, H or R;
X₂ is H, R or K;
X₃ is G, A or P;
X₄ is L, R, I or V;
X₅ is Y, F or M;
X₆ is N or Q;
X₇ is L, V or I;
X₈ is K, R or H;
X₉ is Q, N or S;
X₁₀ is C;
X₁₁ is K, H or R;
X₁₂ is M, F, W or Y;
X₁₃ is S T, N or Q;
X₁₄ is L, V or I;
X₁₅ is N, Q or S;
X₁₆ is G, A, S or P;
X₁₇ is Q, N, S or T; and
X₁₈ is R, K or H.

In a further aspect, the present invention provides an isolated peptide comprising the amino acid sequence: X₁ X₂ X₃ X₄ X₅ X₆ X₇ X₈ X₉ X₁₀ X₁₁ X₁₂ X₁₃, wherein:
X₁ is K, H or R;
X₂ is H, R or K;
X₃ is G, A or P;
X₄ is L, R, I or V;
X₅ is Y, F or M;
X₆ is N or Q;
X₇ is L, V or I;
X₈ is K, R or H;
X₉ is Q, N or S;
X₁₀ is C;
X₁₁ is K, H or R;
X₁₂ is M, F, W or Y; and
X₁₃ is S, T, N or Q.

Thus, the present invention provides a peptide that can comprise, consist essentially of or consist of 18 amino acids defined as X₁ through X₁₈ (i.e., an 18-mer peptide), a peptide that can comprise consist essentially of or consist of 17 amino acids defined as X₁ through X₁₇ (i.e., a 17-mer peptide), a peptide that can comprise, consist essentially of or consist of 16 amino acids defined as X₁ through X₁₆ (i.e., a 16-mer peptide), a peptide that can comprise, consist essentially of or consist of 15 amino acids defined as X₁ through X₁₅ (i.e., a 15-mer peptide), a peptide that can comprise, consist essentially of or consist of 14 amino acids defined as X₁ through X₁₄ (i.e., a 14-mer peptide), a peptide that can comprise, consist essentially of or consist of 13 amino acids defined as X₁ through X₁₃ (i.e., a 13-mer peptide) and a peptide that can comprise, consist essentially of or consist of 12 amino acids defined as X₁ through X₁₂ (i.e., a 12-mer peptide).

The present invention also provides a) a peptide comprising, consisting essentially of or consisting of the amino acid sequence KHGLYNLKQCKMSLNGQR; b) a peptide comprising the amino acid sequence KHGLYNLKQCKMSLNGQR, wherein the K at position 1 is substituted with R or H, the H at position 2 is substituted with R or K, the K at position 8 is substituted with R or H, the K at position 11 is substituted with R or H, the R at position 18 is substituted with K or H, in any combination; or c) a pharmaceutically acceptable salt of any of (a) or (b) above, wherein the peptide is not a full length insulin-like growth factor binding protein (IGFBP-2).

In some embodiments, the peptide of this invention can comprise a polyalkylene glycol moiety coupled to the N terminus thereof, the C terminus thereof, or both the N terminus and C terminus thereof. The polyalkylene glycol moiety can, in some embodiments, be polyethylene glycol (PEG). In some embodiments, the PEG can have a molecular weight from about 10,000 g/mol to about 30,000 g/mol.

In some embodiments, the isolated peptide can comprise nonnatural amino acids as are known in the art to stabilize the peptide. In some embodiments, the isolated peptide can be modified according to methods known in the art to increase the plasma residence time (e.g., extend the half life) of the peptide. See, for example, Pollaro and Heinis “Strategies to prolong plasma residence time of peptide drugs” Med Chem Commun 1:319-324 (2010), the entire contents of which are incorporated by reference herein.

Also provided herein is a composition (e.g., a pharmaceutical formulation) comprising the isolated peptide of this invention in a pharmaceutically acceptable carrier.

In some embodiments, the methods of administering a peptide or composition of this invention to a subject can further comprise administering a weight control agent to the subject, before, after and/or simultaneously with the administration of the peptide or composition. Nonlimiting examples of a weight control agent of this invention include of an appetite suppressant, a lipase inhibitor, an antidepressant, an anti-seizure agent, an anti-preadipocyte differentiation factor and any combination thereof, as are known in the art.

Also provided herein are methods that employ the peptides and compositions of this invention. Thus, the present invention provides a method for controlling body weight and/or treating obesity in a subject (e.g., a subject in need thereof), comprising administering to said subject the peptide and/or composition of this invention in an amount effective to control body weight and/or treat obesity.

In some embodiments of the method for controlling body weight and/or treating obesity in a subject, the peptide or composition comprising the peptide can be administered to the subject concurrently with one or more weight control agents and/or before and/or after administration of one or more weight control agents.

Also provided herein is a method of inhibiting weight gain in a subject, comprising administering to the subject an effective amount of a peptide or composition of this invention.

Additionally provided herein is a method of inhibiting fat development in a subject, comprising administering to the subject an effective amount of a peptide or composition of this invention.

Further provided herein is a method of reducing weight in a subject, comprising administering to the subject an effective amount of a peptide or composition of this invention.

Furthermore, the present invention provides a method of reducing fat mass in a subject, comprising administering to the subject an effective amount of a peptide or composition of this invention,

Additionally provided herein is a method of treating obesity in a subject, comprising administering to the subject an effective amount of a peptide or composition of this invention.

In some embodiments of this invention, the subject has insulin resistance and in some embodiments of this invention, the subject does not have insulin resistance.

In the methods of this invention, the amount of the peptide and/or composition of this invention is an amount effective to reduce weight gain or induce weight loss in said subject and/or an amount effective in reducing the body mass index of said subject.

In some embodiments of the methods of this invention, the subject can have a body mass index of at least about 25 kg/m² and in some embodiments, the subject can have a body mass index of at least about 30 kg/m².

In some embodiments of this invention, the peptide and/or composition can be administered for a period of at least about 16 weeks or about 24 weeks.

In some embodiments of the methods of this invention, the peptide and/or composition can be administered until said subject has achieved at least 5% weight loss or said subject's body mass index is reduced to less than about 25 kg/m²,

In some embodiments of the methods of this invention, the peptide and/or composition is administered in an amount that is effective in inducing fat loss in said subject and/or in an amount that is effective in inhibiting fat cell differentiation (e.g., inhibiting fat cell precursor differentiation into mature adipocytes) in said subject. Thus, in additional embodiments, the present invention provides a method of inhibiting fat cell differentiation in a subject, comprising administering to the subject an effective amount of a peptide or composition of this invention.

Further aspects of this invention include the use of the peptide and/or composition of this invention in the preparation of a medicament for carrying out the methods of this invention.

An additional aspect is the use of the peptide and/or composition of this invention for carrying out the methods of this invention.

In some embodiments, the subject is obese or overweight. In some embodiments, the subject is not obese or overweight.

The therapeutically effective amount or dosage of any specific active compound of this invention will vary from compound to compound, and patient to patient, and will depend, among other things, upon the effect or result to be achieved, the condition of the patient and/or the route of delivery. In some embodiments, a dosage from about 0.001 mg/kg (i.e., 1 ug/kg), 0.05, 0.1, 0.2, 0.3. 0.4, 0.5, 0.6, 0.7, 0.8, 0.9 or 1.0 mg/kg, up to about 30, 40 or 50 mg/kg (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 mg/kg), or more, may be used.

Administration of the compound or composition of this invention may be by any suitable route, including intrathecal injection, subcutaneous, cutaneous, oral, intravenous, intraperitoneal, intramuscular injection, nasal, oral, sublingual, via inhalation, in an implant, in a matrix, in a gel, or any combination thereof.

### A. Definitions.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

“Subjects” as used herein include any animal in which body weight control or treatment of obesity is necessary or desired. A subject of this invention can be a subject in need of body weight control or treatment of obesity and thus, in some embodiments, the subject of the methods of this invention can be obese or overweight and/or prone to being obese or overweight. In some embodiments, the subject of this invention can be a subject that desires body weight control or for whom body weight control is desired and such subjects may or may not be obese or overweight. In some embodiments, a subject of this invention can be a mammalian subject, which can be a human subject. A subject of this invention can be male or female and may be of any race or ethnicity, including, but not limited to, Caucasian, African-American, African, Asian, Hispanic, Indian, etc. The subjects may be of any age, including newborn, neonate, infant, child, adolescent, adult, and geriatric. Subjects may also include animal subjects, particularly mammalian subjects such as canines, felines, bovines, caprines, equines, ovines, porcines, rodents (e.g. rats and mice), lagomorphs, primates (including non-human primates), etc., for veterinary medicine or pharmaceutical drug development purposes.

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

“Treat,” “treating” or “treatment” as used herein also refers to any type of action or administration that imparts a benefit to a subject that has a disease or disorder, including improvement in the condition of the patient (e.g., reduction or amelioration of one or more symptoms), healing, etc.

A “treatment effective” amount as used herein is an amount that is sufficient to treat (as defined herein) the subject. Those skilled in the art will appreciate that the therapeutic effects need not be complete or curative, as long as some benefit is provided to the subject.

The terms “therapeutically effective amount” and “effective amount” as used herein are synonymous unless otherwise indicated, and mean an amount of a compound, peptide or composition of the present invention that is sufficient to improve the condition, disease, or disorder being treated and/or achieved the desired benefit or goal (e.g., control of body weight). Determination of a therapeutically effective amount, as well as other factors related to effective administration of a compound of the present invention to a subject of this invention, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this invention is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the term “prevent,”, “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

“Concurrently administering” or “concurrently administer” as used herein means that the two or more compounds or compositions are administered closely enough in time to produce a combined effect (that is, concurrently may be simultaneously, or it may be two or more events occurring within a short time period before or after each other, e.g., sequentially). Simultaneous concurrent administration may be carried out by mixing the compounds prior to administration, or by administering the compounds at the same point in time but at different anatomic sites and/or by using different routes of administration.

“Pharmaceutically acceptable” as used herein means that the compound or composition is suitable for administration to a subject to achieve the treatments described herein, without unduly deleterious side effects in light of the severity of the disease and necessity of the treatment.

“Weight control agent” as used herein includes any weight control agent, including but not limited to an anti preadipocyte differentiation factor; appetite suppressants such as sibutramine, phentermine, diethylpropion, phendimetrazine, etc.; lipase inhibitors such as orilstat; antidepressants such as bupropion; anti-seizure agents such as topiramate, zonisamide, and metformin, etc., as are known in the art.

“Polyalkylene oxides” as used herein are known (see, e.g., U.S. Pat. No. 7,462,687) and include poly(ethylene glycol) or “PEG”. Additional examples may contain hetero atoms such as S or N, and are typically linear polyalkylene oxides such as: O—(CH₂CH₂O)ₓ—, —O—C(O)CH₂—O—(CH₂CH₂O)ₓ—CH₂C(O)—O—, —NRCH₂CH₂2—O—(CH₂CH₂O)ₓ—CH₂CH₂NR—, and —SHCH₂CH₂—O—(CH₂CH₂O)ₓ—CH₂CH₂SH—, wherein R is H or lower alkyl (preferably methyl), and x is an integer that provides or yields a total number average molecular weight for the molecule of from about 1,000, 2,000, 3,000, 4,000, 5,000, 6,000, 7,000, 8,000, 9,000, 10,000, 15,000, 20,000, 30,000, 40,000, 50,000, 60,000, 70,000, 80,000, 90,000 or 100,000 daltons or more.

“Analog” as used herein is a peptide that has the physiological activity of the parent compound thereof, and that includes one or more (e.g., two, three, four, five or six or more) amino acids different from the amino acid sequence of a naturally occurring parent peptide. Such an analog preferably has at least about 70% of the physiological activity of the parent peptide. Such different amino acids may be additions, substitutions, deletions, or combinations thereof, including addition of non-natural side-chain groups and backbone links. Modifications of peptides to produce analogs thereof are known. See, e.g., U.S. Pat. No. 7,323,543; see also U.S. Pat. Nos. 7,482,171; 7,459,152; and 7,393,919.

### B. Active Compounds.

The single letter code for amino acids as used herein is: A (Ala), C (Cys), D (Asp), E (Glu), F (Phe), G (Gly), H (His), I (Ile), K (Lys), L (Leu), M (Met), N (Asn), P (Pro), Q (Gln), R (Arg), S (Ser), T (Thr), V (Val), W (Trp), and Y (Tyr)).

The amino acid sequence of human insulin-like growth factor binding protein-2 (IGFBP-2) is shown below. The peptide KHGLYNLKQCKMSLNGQR is bolded in the sequence below.

| | |
|---|---|
| 1 | mlprvgcpalplppppllpllpllllllgasgggggaraevlfrcppctperlaacgppp |
| 61 | vappaavaavaggarmpcaelvrepgcgccsvcarlegeacgvytprcgqglrcyphpgs |
| 121 | elplqalvmgegtcekrrdaeygaspeqvadngddhsegglvenhvdstmnmlggggsag |
| 181 | rkplksgmkelavfrekvteqhrqmgkggkhhlgleepkklrpppartpcqqeldqvler |
| 241 | istmrlpdergplehlyslhipncdkhglynlkqckmslngqrgecwevnpntgkliqga |
| 301 | ptirgdpechlfyneqqeargvhtqrmq |

Nonlimiting examples of an active compound of this invention include a peptide comprising, consisting essentially of or consisting of the amino acid sequence KHGLYNLKQCKMSLNGQR, a peptide comprising, consisting essentially of or consisting of the amino acid sequence KHGRYNLKQCKMSLNGQR, and a peptide comprising, consisting essentially of or consisting of the amino acid sequence KHGLYNLKQCKMSLNGQR (18 amino acids shown in this peptide are numbered 1 through 18, consecutively from N terminus to C terminus), wherein the K at position 1 is substituted with R or H, the H at position 2 is substituted with R or K, the K at position 8 is substituted with R or H, the K at position 11 is substituted with R or H, the R at position 18 is substituted with K or H, in any combination; or a prodrug, analog and/or pharmaceutically acceptable salt of any of the peptides shown above. The peptide of this invention is not a full length insulin-like growth factor binding protein 2 (IGFBP-2).

Amino acids in peptides of the present invention may be in the D or L configuration: e.g., all D; all L; some D and some L in any combination.

Nonlimiting examples of an active compound of this invention include the peptides of this invention, analogs thereof, a prodrug of any thereof, or pharmaceutically acceptable salts of any thereof.

Additional examples of compounds of the present invention include any or all of the foregoing compounds, where the first one, two, three, four or five amino terminal amino acids are deleted and/or the first one, two, three or four C-terminal amino acids are deleted.

Additional examples of compounds of the present invention include any or all of the foregoing compounds, with one, two three, four or five additional carboxy terminal amino acids of any type and/or N-terminal amino acids of any type coupled thereto.

Additional examples of compounds of the present invention include any or all of the foregoing compounds, with a 10,000 to 30,000 molecular weight of poly(ethylene glycol) (or “PEG”) moiety coupled to either the N or C terminus thereof or both the N terminus and C terminus.

“Polyalkylene glycol” means straight or branched polyalkylene glycol polymers including, but not limited to, polyethylene glycol (PEG), polypropylene glycol (PPG), and polybutylene glycol (PBG), as well as co-polymers of PEG, PPG and PBG in any combination, and includes the monoalkylether of the polyalkylene glycol. Thus, in various embodiments of this invention, the polyalkylene glycol in the compositions of this invention can be, but is not limited to, polyethylene glycol, polypropylene glycol, polybutylene glycol, and any combination thereof.

In certain embodiments, the polyalkylene glycol of the composition is polyethylene glycol or “PEG.” The term “PEG subunit” refers to a single polyethylene glycol unit, i.e., —(CH₂CH₂O)—.

In some embodiments, the polyalkylene glycol (e.g., PEG) can be non-polydispersed, monodispersed, substantially monodispersed, purely monodispersed, or substantially purely monodispersed.

“Monodispersed” is used to describe a mixture of compounds wherein about 100 percent of the compounds in the mixture have the same molecular weight.

“Substantially monodispersed” is used to describe a mixture of compounds wherein at least about 95 percent of the compounds in the mixture have the same molecular weight.

“Purely monodispersed” is used to describe a mixture of compounds wherein about 100 percent of the compounds in the mixture have the same molecular weight and have the same molecular structure. Thus, a purely monodispersed mixture is a monodispersed mixture, but a monodispersed mixture is not necessarily a purely monodispersed mixture.

“Substantially purely monodispersed” is used to describe a mixture of compounds wherein at least about 95 percent of the compounds in the mixture have the same molecular weight and have the same molecular structure. Thus, a substantially purely monodispersed mixture is a substantially monodispersed mixture, but a substantially monodispersed mixture is not necessarily a substantially purely monodispersed mixture.

The bioavailability of compounds targeted to intracellular sites depends on the requirements of being sufficiently polar for administration and distribution, yet non-polar enough to diffuse through the non-polar lipid bilayer of the cell (Begley, Journal of Pharmacy & Pharmacology 48:136-146 (1996)). A strategy for delivery of synthetic compounds across cell membranes has been investigated by both industry and academic researchers (R. Service, Science 288:28-29 (2000)). Positively charged, cationic peptides are known to cross cell membranes independent of receptors or specific transport mechanisms (Schwarze et al., Science 285:1569-1572 (1999); Ho et al., Cancer Research 61:474-477 (2001); Morris et al., Nature Biotechnology 19:1173-1176 (2001); Pooga et al., FASEB Journal 12:67-77 (1998); Derossi et al., Journal of Biological Chemistry 271:18188-18193(1996); Pietersz et al., Vaccine 19:1397-1405 (2001); Elliott and O'Hare, Cell 88:223-233 (1997); Derer et al., FASEB Journal 16:132-133 (2002); Will et al., Nucleic Acids Research 30:e59 (2002); Rothbard et al., Journal of Medicinal Chemistry 45:3612-3618 (2002); Chen et al., Chemistry & Biology 8:1123-1129 (2001); Wender et al., Proceedings of the National Academy of Sciences of the United States of America 97:13003-13008 (2000)). The transport involves protein transduction domains (PTDs) that are highly charged, short peptides (˜10 to 20 amino acids), containing basic amino acids (arginines and lysines), and that have the ability to form hydrogen bonds. The ability of PTDs to cross cell membranes is also concentration-dependent.

Attachment of nucleic acids, peptides, and even large proteins to these PTDs will allow their transduction across all cell membranes in a highly efficient manner (Schwarze and Dowdy, Trends in Pharmacological Sciences 21:45-48 (2000)). Three PTDs have been described which share the common characteristics of being potential DNA binding proteins: HIV-TAT, VP22, and Antennapedia (Schwarze et al., Science 285:1569-1572 (1999); Derossi et al., Journal of Biological Chemistry 271:18188-18193(1996); Elliott and O'Hare, Cell 88:223-233 (1997).

The PTD (e.g., cell penetrating peptide (CPP)) derived from the HIV genome, HIV-TAT (trans-activator of transcription, “TAT”), has the ability to move attached peptides, large proteins, and nucleic acids across virtually all cell membranes, including brain, in a non-receptor mediated fashion (Schwarze et al., Science 285:1569-1572 (1999); Cao et al., Journal of Neuroscience 22:5423-5431 (2002); Gustafsson, et al., Circulation 106:735-739 (2002); Nagahara et al., Nature Medicine 4:1449-1452 (1998)). The attached proteins are refolded into an active confirmation once inside the cell and are biologically active. The full length TAT protein, originally described in 1988, by Green and Lowenstein, is an 86 amino acid protein encoded by the HIV virus (Fawell et al., Proc. Natl. Acad. Sci. U.S.A. 91:664-668 (1994); Frankel, and Pabo, Cell 55:1189-1193(1988); Green and Loewenstein, Cell 55:1179-1188(1988)). More specifically, an 11 amino acid arginine-and lysine-rich portion of the TAT sequence, YGRKKRRQRRR, conjugated to peptides that do not normally cross membranes, is able to transduce across cell membranes and deliver a biologically active fusion protein to tissues. Furthermore, when a TAT-fusion protein was injected into mice for two weeks, there were no gross signs of neurological problems or system distress. Previously, TAT-fusion proteins were shown to be capable of delivering an active fusion protein that affects mitochondrial function, though in both cases, the fusion protein was not processed by the mitochondria. (Cao et al., Journal of Neuroscience 22:5423-5431 (2002); Gustafsson et al., Circulation 106:735-739 (2002)).

In some embodiments, the peptides of this invention are cyclized. Cyclization of peptides is well known in the art (see, e.g., U.S. Pat. No. 4,102,877, US Patent Publication No. 2008/0097079, the entire contents of each of which is incorporated by reference herein). As one nonlimiting example, a peptide can be cyclized on a solid support (e.g., resin). A variety of cyclization reagents can be used such as HBTU/HOBt/DIEA, PyBop/DIEA, PyClock/DIEA. Head-to-tail peptides can be made on the solid support. The deprotection of the C-terminus at some suitable point allows on-resin cyclization by amide bond formation with the deprotected N-terminus. Once cyclization has taken place, the peptide is cleaved from resin by acidolysis and purified. The strategy for the solid-phase synthesis of cyclic peptides in not limited to attachment through Asp, Glu or Lys side chains. Cysteine has a very reactive sulfhydryl group on its side chain. A disulfide bridge is created when a sulfur atom from one cysteine fauns a single covalent bond with another sulfur atom from a second cysteine in a different part of the protein. These bridges help to stabilize proteins, especially those secreted from cells. Modified cysteines can be employed, using S-acetomidomethyl (Acm) to block the formation of the disulfide bond but preserve the cysteine and the protein's original primary structure.

C. Pharmaceutical Formulations. The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

Furthermore, a “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include any of the standard pharmaceutical carriers such as phosphate buffered saline solutions, water, emulsions such as oil/water emulsion, microemulsions and various types of wetting agents.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

In some embodiments of this invention, the peptide or compound of this invention is present in an aqueous solution for subcutaneous administration. In some embodiments, the peptide or compound is provided as a lyophilized powder that is reconstituted and administered subcutaneously.)

The present invention is illustrated in the following non-limiting examples.

### EXAMPLES

### Example 1

### Materials and Methods

Immobilon-P membrane was purchased from Millipore Corp. (Bedford, Mass.). Dulbecco's modified Eagle medium (DMEM) containing 4,500 mg glucose per liter (25 mM), streptomycin, and penicillin were purchased from Gibco (Grand Island, N.Y.). The horseradish peroxidase (HRP)-conjugated mouse anti-rabbit and goat anti-mouse antibodies were purchased from Jackson ImmunoResearch Laboratories (West Grove, Pa.). IGFBP-2 antiserum was prepared as previously described (Cohick W S, Clemmons D R, 1991). All other reagents were purchased from Sigma Chemical Company (St. Louis, Mo.) unless otherwise stated.

### Generation of Synthetic Peptides and Peptides Pegylation

The synthetic peptide containing the linker located heparin-binding domain of mouse IGFBP-2 (KHLSLEEPKKLRP) (referred to as HBD1 peptide) was prepared. A scrambled HBD1 peptide (CKPLRLSKEEHPLK) (referred to as HBD1 control peptide) was prepared. A peptide containing the C-terminal heparin-binding domain of human IGFBP-2 (KHGLYNLKQCKMSLNGQR) (referred to as HBD2 peptide) was also prepared. A peptide containing the C-terminal heparin-binding domain of IGFBP-5 (RKGFYKRKQCKPSRGRKR) (referred to as HBD2 control peptide) was prepared. The peptides were synthesized by the Protein Chemistry Core Facility at the University of North Carolina at Chapel Hill. Purity and the sequences were confirmed by mass spectrometry.

The mouse IGFBP-2 amino acid sequence (GenBank® Database Accession No. AAB60709) is shown below.

| | |
|---|---|
| 1 | mlprlggpal plllpsllll lllgaggcgp gvraevlfrc ppctperlaa cgpppdapca |
| 61 | elvrepgcgc csvcarqege acgvyiprca qtlrcypnpg selplkalvt gagtcekrrv |
| 121 | gttpqqvads ddhhsegglv enhvdgtmnm lgggssagrk plksgmkela vfrekvneqh |
| 181 | rqmgkgakhl sleepkklrp ppartpcqqe ldqvlerist mrlpddrgpl ehlyslhipn |
| 241 | cdkhgrynlk gckmslngqr gecwcvnpnt gkpiggapti rgdpechlfy neqqetggah |
| 301 | aqsvq |

The human IGFBP-5 amino acid sequence (GenBank® Database Accession No. NP_{—}000590) is shown below.

| | |
|---|---|
| 1 | mvlltavlll laayagpaqs lgsfvhcepc dekalsmcpp splgcelvke pgcgccmtca |
| 61 | laegqscgvy tercaqglrc lprqdeekpl hallhgrgvc lneksyreqv kierdsrehe |
| 121 | epttsemaee tyspkifrpk htriselkae avkkdrrkkl tqskfvggae ntahpriisa |
| 181 | pemrqeseqg pcrrhmeasl qelkasprmv pravylpncd rkgfykrkqc kpsrgrkrgi |
| 241 | cwcvdkygmk lpgmeyvdgd fqchtfdssn ve |

The human IGFBP-2 amino acid sequence (GenBank® Database Accession No. AAB22308) is shown below.

| | |
|---|---|
| 1 | mlprvgcpal plppppllpl lpllllllga sgggggarae vlfrcppctp erlaacgppp |
| 61 | vappaavaav aggarmpcae lvrepgcgcc svcarlegea cgvytprcgq glrcyphpgs |
| 121 | elplqalvmg egtcekrrda eygaspeqva dngddhsegg lvenhvdstm amlggggsag |
| 181 | rkplksgmke lavfrekvte qhrqmgkggk hhlgleepkk lrpppartpc qqeldqvler |
| 241 | istmrlpder gplehlyslh ipncdkhgly nlkqckmsln gqrgecwcvn pntgkliqga |
| 301 | ptirgdpech lfyneqqear gvhtqrmq |

HBD1, HBD2 and HBD1 control peptides that had a cysteine added to their C-terminus were synthesized and then pegylated as follows: 10 mg of peptide was mixed with 380 μg of methoxy PEG maleimide (20000 kDa) (1:3 molar ratio peptide to PEG) (JenKem Biotechnology, Allen, Tex.) in 4.0 ml of 0.05M NaPO₄ (pH 7.0). Following an overnight incubation at 4° C., cysteine was added to a final concentration of 17 mM to block untreated sites. To remove the non-pegylated peptide and cysteine, the mixture was desalted using a Zebra Desalt spin column (Thermo Scientific, Rockford, Ill.) following the manufacturer's instructions. Pegylation was verified by SDS-PAGE analysis with Coomassie staining.

Generation of pLenti-IGFBP-2 Wild Type (WT) and Two HBDs Mutants

Mouse IGFBP-2 cDNA was amplified from mouse pCMV-SPORT6 (ATCC, Manassas, Va.) using a 5′ primer sequence corresponding to nucleotides 89 to 110 of mouse IGFBP-2 (5′-ATGCTGCCGAGATTGGGCGGCC-3′) and a 3′ primer sequence complementary to nucleotides 981 to 1003 (5′-GGGCCCATGCCCAAAGTGTGCAG-3′). After DNA sequencing to confirm that the correct sequence had been amplified, the PCR product was subcloned into pENTR/D-TOPO vector and subsequently transferred into the pLenti6-V5 DEST expression vector using the LR Clonase reaction and following the manufacturer's instructions (Invitrogen, Carlsbad, Calif.).

The wild-type IGFBP-2 inserted into the pENTR/D-TOPO vector was used as a template to make the substitution mutant. The two IGFBP-2 mutants incorporated substitutions of amino acids within the HBD1 domain of mouse IGFBP-2 containing the sequence KHLSLEEPKKLRP and HBD2 domain of mouse IGFBP-2 containing the sequence KHGRYNLKQCKMSLNGQR. The substitutions, highlighted in bold, were as follows: AALSLEEPAALA (referred to as HBD1 mutant) and AAGRYNAAQCAMSLNGQA (referred to as HBD2 mutant), respectively. The QuikChange site-directed mutagenesis kit by Stratagene (Agilent Technologies, Santa Clara, Calif.) was used to incorporate the base changes needed to encode these substitutions (shown in bold below).

To generate the HBD1 mutant construct, the following primer was used: 5′-AAGGGTGCCGCAGCCCTCAGTCTGGAGGAGCCCCGCGGCGTTGGCCCCGCCTCC C-3′. To generate the HBD2 mutant construct, two rounds of PCR were performed. The following primer was used for first round PCR: 5′-AACTGTGACGCGGCTGGCCGGTACAACGCGGCGCAGTGCAAGATG-3′. After confirming that the correct sequences were present, the first round PCR product was used as a template to perform second round PCR. The following primer was used for second round PCR: 5′-AACGCGGCGCAGTGCGCGATGTCTCTGAACGGACAGGCGGGGGAGTGCTGG-3′.

After selection of the correct clone based on sequence analysis, the cDNAs encoding the two mutated forms of IGFBP-2 were transferred from pENTR/D-TOPO vector into pLenti6-V5 DEST vector using the LR Clonase reaction according to the manufacturer's instructions (Invitrogen, Carlsbad, Calif.).

### Preparation of Viral Stocks

Viral stocks were generated with 293FT cells (Invitrogen, Carlsbad, Calif.) for each individual pLenti-construct. Cells were plated at 5×10⁶ per 75-cm² plate the day before transfection in the growth medium (DMEM-H with 10% FBS, streptomycin at 100 and penicillin at 100 U/ml). On the day of transfection, the culture medium was replaced with 5 ml of Opti-modified Eagle's medium-I (Invitrogen, Carlsbad, Calif.) without antibiotics and serum. DNA-Lipofectamine 2000 complexes for each transfection were prepared according to the manufacturer's protocol (Invitrogen, Carlsbad, Calif.). The next day, the medium containing the DNA-Lipofectamine 2000 complexes was removed and replaced with the growth medium. The virus-containing supernatants were harvested 48-72 h after transfection and centrifuged at 3000 rpm for 15 min at 4° C. to pellet the cell debris. The supernatants were filtered and stored as 1-ml aliquots at −80° C.

### Purification of Wild Type and Two HBD Mutant Forms IGFBP-2

The constructs were expressed in CHO-K1 cells by using the procedure described previously (Gockerman A, 1995). Conditioned medium was collected from confluent CHO-K1 cells expressing WT IGFBP2 or the HBD1 mutant or HBD2 mutant that had been maintained in serum-free α-MEM for 48 h. The expressed proteins were purified following the procedure described previously (Shen et al., 2013).

### Cell Culture of Primary Preadipocytes

Primary preadipocytes were isolated from epididymal fat pads of 6 week old IGFBP-2^{-/-} mice. The cells were cultured using a previously described method (Boney, C M et al., 1994). The fat pads were weighed and the tissue was digested with an equal volume (W/V) of collagenase type A (Roche Applied Science, Indianapolis, Ind.) in HEPES buffer (0.1M and 1.5% BSA) for 45 min at 37° C. Following complete digestion, the preadipocytes were separated from the stromal vascular cells by centrifugation at 500×g for 5 min. The pelleted cells were washed with DMEM containing 30 mM glucose (Gibco, Grand Island, N.Y.) and passed through a 100 μm mesh filter (BD Biosciences, Bedford, Mass.). The cells were then plated in 6-well plates (Falcon Corp., Franklin Lakes, N.J.) at a density of 1.2-1.5×10⁴ cells/ml in DMEM containing 30 mM glucose supplemented with penicillin (100 U/ml), streptomycin (100 μg/ml), and 10% fetal bovine serum hereafter referred to as standard medium. The cultures had the medium changed every 2 days until they reached confluency. Two day post-confluent cells were then exposed to differentiation medium (serum free DMEM containing 0.5 mM IBMX, 1 μM dexamethasone, and 5 μg/mL insulin) and incubated for 2 days. Experimental treatments that were added to this medium included either wild type IGFBP-2 (3 μg/ml), HBD1 peptide (6 μg/ml), HBD1 control peptide (6 μg/ml), HBD2 peptide (6 μg/ml), HBD2 control peptide (6 μg/ml), HBD1 mutant IGFBP-2 (3 μg/ml) or the HBD2 mutant IGFBP-2 (3 μg/ml). Cultures were maintained for two additional days in the standard medium supplemented with 5 μg/ml insulin. Thereafter, the cultures were kept in the fresh standard medium without insulin for additional two days.

### Oil Red O Staining

After treatment, the cells were rinsed with PBS for two times and fixed with 10% formalin for 30 min at room temperature. After rinsing with distilled water for twice, 100% propylene glycol (Poly Scientific, Bay Shore, N.Y.) was added and incubated for 5 min. Before adding Oil Red O solution (Poly Scientific, Bay Shore, N.Y.), propylene glycol was removed. The cultures were incubated for 10 min at 60° C. Removing Oil Red O solution, the cultures were incubated with 80% propylene glycol for 5 min, After incubation, the cultures were rinsed with tap water twice. Images were captured using an Olympus IX81 inverted microscope.

### Immunoprecipitation and Immunoblotting

Differentiated adipocytes were lysed in ice-cold lysis buffer (Xi et al., 2008). After centrifugation at 14,000×g for 10 min, solubilized proteins were quantified by the Bradford method (Thermo Scientific, Rockford, Ill.). Equal protein amounts of lysates were loaded onto a SDS-polyacrylamide gel and the proteins were separated, then transferred to an Immobilon filter and visualized by immunoblotting using the appropriate antibody. The primary antibody dilutions that were used were 1:1000 for the anti-adiponectin antibody (Affinity BioReagents, Golden, Colo.), 1:500 for the anti-PPARγ antibody (Cell Signaling Technology Inc., Beverly, Mass.), 1:2000 for the anti-apt antibody (ProSci Inc., Poway, Calif.) and 1:5000 for the anti-β-actin antibody (Sigma Chemical Company, St. Louis, Mo.). The immune complexes were visualized using enhanced chemiluminescence (Thermo Fischer Scientific, Rockford, Ill.).

### Mice

Generation of the B6.129-Igfbp2^{tmlJep}, referred to as Igfbp2^{-/-} mice, has been described previously (DeMambro et al., 2008; Danno et al., 1992). The mice were backcrossed onto C57BL/6J background for at least 10 generations. Mating pairs were provided from Maine Medical Center Research Institute (Scarborough, Me.). Igfbp2^{+/+} mice were C57BL/6J controls. All in vivo and ex vivo experimental studies were performed using male mice. The animal study protocol was reviewed and approved by the Institutional Animal Care and Use Committee of University of North Carolina at Chapel Hill. Mice were assigned to one of four treatment groups. 1) Peg HBD1 peptide: Igfbp2^{-/-} mice were administered 50 μg of the pegylated HBD1 peptide in 0.1 mL PBS (n=9); 2) Peg HBD2 peptide: Igfbp2^{-/-} mice were administered 50 μg of the pegylated HBD2 peptide in 0.1 mL PBS (n=10); 3) control peptide: Igfbp2^{-/-} mice were given 50 μg of the HBD1 control peptide in 0.1 mL PBS (n=18); 4) WT: Igfbp2^{+/+} mice were given 0.1 mL of PBS (n=9). All injections were administered IP 3 times weekly from 10-22 weeks of age. All mice were provided with free access to 2018 Teklad global rodent diet (Harlan, Dublin, Va.), containing 18.6% protein, 6.2% fat and 3.5% crude fiber. Food consumption and the weights of the mice were determined weekly.

### Body Composition and Serum Adipokines Measurement

Body fat and lean muscle mass was measured at weeks 0 and 12 of treatment by magnetic resonance imaging analysis (EchoMRI-100, Echo Medical Systems, Houston, Tex.), a technique that measures fat mass and lean mass in unanesthetized animals (Kelly S A, et. al., 2010). After completion of 12 weeks of treatment, the mice were sacrificed. Blood was collected by cardiac puncture and allowed to sit for 4 hours before centrifuging at 3500 rpm×15 min. The serum was aspirated and stored at −20° C. until analysis. Abdominal inguinal and visceral fat pads were carefully dissected from each animal according to defined anatomical landmarks. All subcutaneous fat between the lower part of the rib cage and the upper thigh was considered abdominal subcutaneous inguinal fat, whereas all fat found along the mesentery starting at the lesser curvature of the stomach and ending at the sigmoid colon was considered visceral fat. Fat depots were blotted dry prior to weighing. The right inguinal fat pad was dissected separately and the triglyceride content of this tissue was determined by a colorimetric analysis (Pointe Scientific, Canton, Mich.), as previously described (Dann H, Kimura S 1992). Serum adiponectin and leptin were measured by ELISA following manufacturer's instructions (Millipore, Billerica, Mass.).

### Glucose Tolerance Test

Glucose tolerance tests were performed after a 6-hour fast. For oral glucose tolerance tests, mice were given 20% glucose (2.5 g/kg body weight) by oral gavage and blood was obtained from the tail vein for determination of glucose at baseline, 15, 30, 60, 90 and 120 min (Bayer Contour Glucometer, Tarrytown, N.Y.) after gavage.

### Statistical Analysis

All data are expressed as the mean standard error (SEM). Results were analyzed for statistically significant differences using Student's t-test for the data obtained from in vitro assays or ANOVA followed by Bonferroni multiple comparison post hoc test for the data obtained from in vivo assays. Statistical significance was set at p<0.05.

### IGFBP-2 and Peptides Containing its Unique and C-Terminal Heparin Binding Domains Inhibit the Differentiation of Primary IGFBP-2 Preadipocytes.

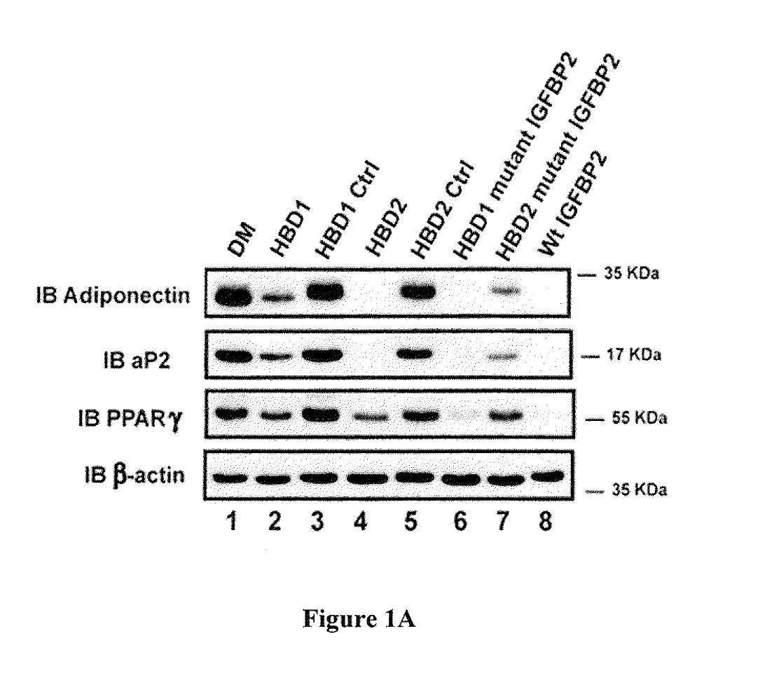

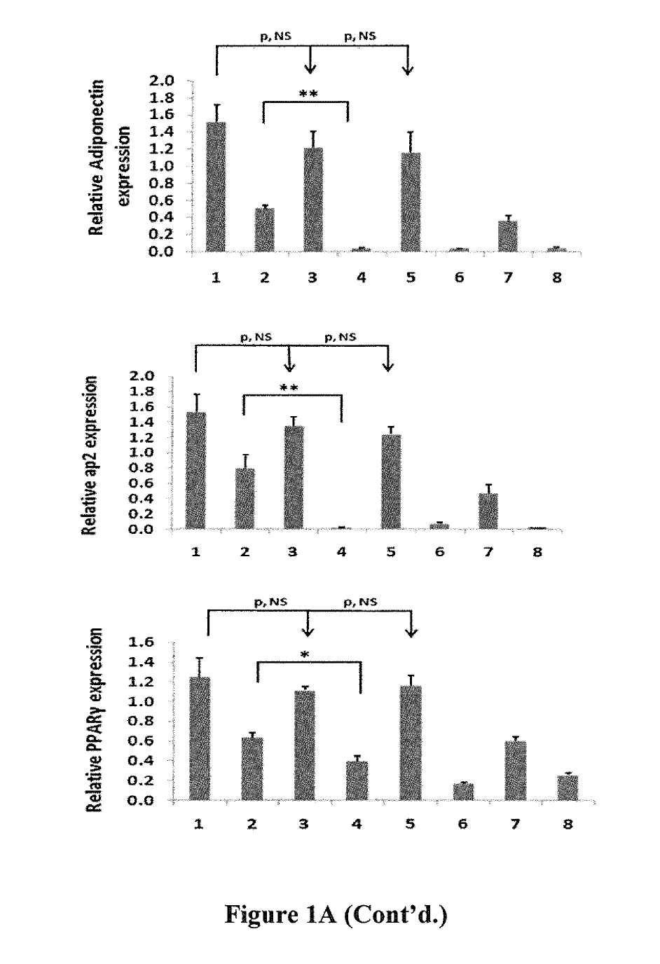

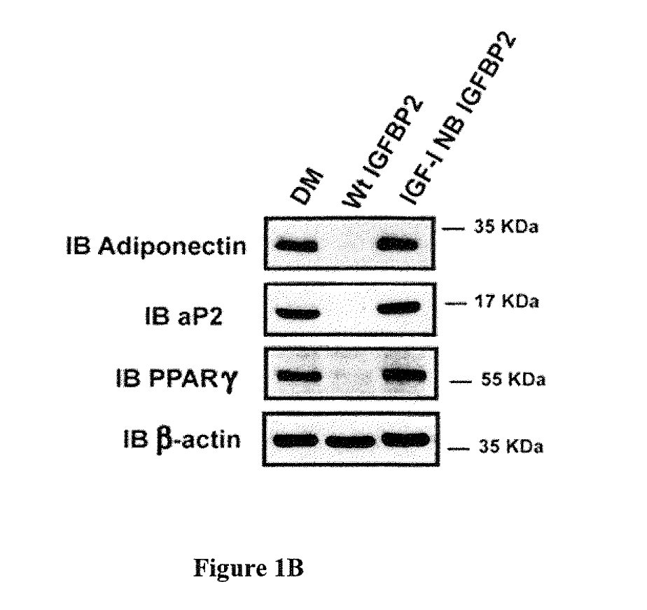

To investigate the effects of each of the heparin binding domains on adipogenesis, peptides that contained the HBD sequences located in the linker region (HBD1) or C-terminal region (HBD2) of IGFBP-2 (FIG. 1A) were synthesized and incubated with cultures of preadipocytes that had been isolated from IGFBP-2 null mice. The results showed that native IGFBP-2 inhibited preadipocyte differentiation into mature adipocytes as indicated by suppression of three differentiation markers adiponection, aP2 and PPARγ (FIG. 1B, lane 8). A peptide containing HBD1 sequence significantly inhibited the expression of adiponectin (e.g. 66±10% reduction, p<0.01), aP2 (e.g. 51±2% reduction, p<0.01) and PPARγ (e.g. 47±9% reduction, p<0.05) (FIG. 1B, lane 2 vs. lane 1). However, a peptide containing a scrambled HBD1 sequence had no effect (FIG. 1B, lane 3 vs. lane 1). Interestingly, a peptide containing HBD2 sequence completely inhibited the expression of adiponectin and aP2 and PPARγ expression was reduced 67 7% (p<0.01) compared to the cells exposed to differentiation medium only (FIG. 1B, lane 4 vs. lane 1). A peptide containing the homologous region of IGFBP-5 sequence, served as a control and it did not affect their expression (FIG. 1B, lane 5 vs. lane 1).

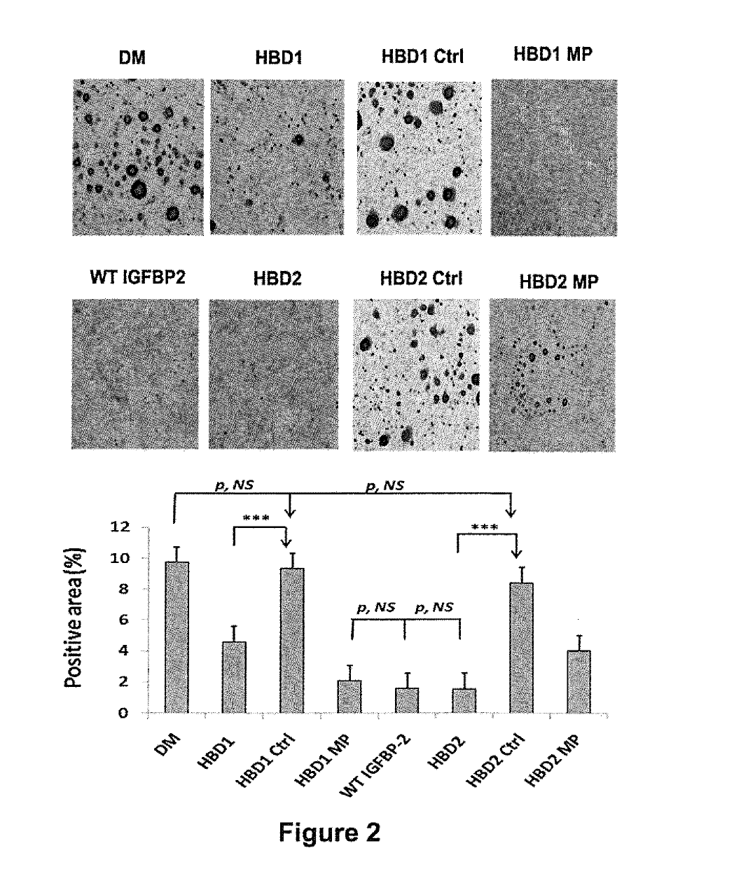

To confirm these results IGFBP-2 mutants in which the charged residues in either the HBD1 or HBP2 domains were changed to neutral residues were prepared and expressed. The IGFBP-2 mutant containing the altered HBD1 sequence but an intact HBD2 sequence inhibited differentiation marker expression as well as wild type IGFBP-2 (FIG. 1B, lane 6 vs. lane 8) and the peptide containing HBD2 sequence (FIG. 1B, lane 6 vs. lane 4). In contrast the IGFBP-2 mutant containing the altered HBD2 residues but an intact HBD1 sequence was significantly less effective in preventing preadipocyte differentiation (FIG. 1B, lane 7 vs. lane 2). When the ability to form differentiated adipocytes was determined using oil red O staining, similar results were obtained (FIG. 2). These results demonstrate that native IGFBP-2, the HBD1 mutant and the HBD2 peptide were the most potent inhibitors of adipogenesis whereas the HBD1 peptide and HBD2 mutant has less activity. Taken together the results strongly suggest that the inhibitory effect of IGFBP-2 on the differentiation of preadipocytes is primarily mediated via its HBD2 domain.

### The Heparin Binding Domains of IGFBP-2 Decrease Weight Gain in IGFBP-2^{-/-} Mice.

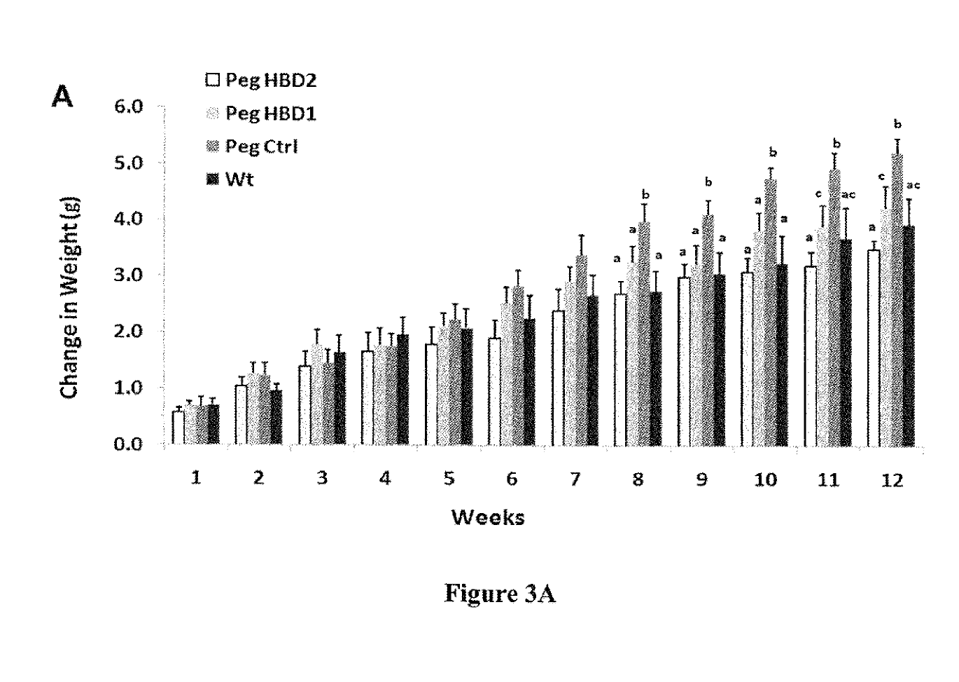

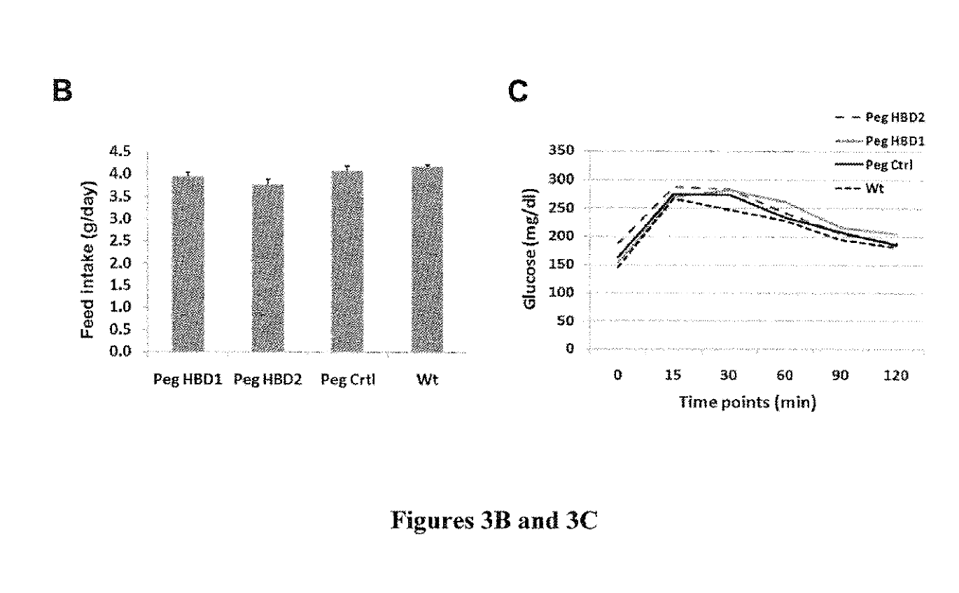

To investigate the effect of IGFBP-2 HBDs on weight gain and fat mass in vivo, the peptides containing HBD sequences were administered to IGFBP-2−/− mice. Since both control peptides had a similar effect on preadiocyte differentiation in vivo, only the scrambled HBD1 peptide was used as a control. To extend the half life of the peptides and increase their resistance to proteolysis in vivo, all three peptides were pegylated. It has been shown that 50 μg of HBD1 peptide administered via intraperitoneal (IP) injection 5 times per week for 3 weeks was effective in promoting bone growth in IGFBP-2^{-/-} male mice (17). This protocol was modified to accommodate a longer period of study and injected 50 μg of each peptide (IP) 3 times/week for 12 weeks. Age matched wild-type C57BL/6J (Wt) male mice were treated with vehicle and compared to the three groups of mice that received peptide treatments. Since this animal study required a large number of IGFBP-2^{-/-} mice, the experiment was separated into two phases. In the first phase, HBD1 and control peptide treatments as well as wild type control mice were studied. In the second phase the effects of the HBD2 and control peptides were determined. The weights and ages of the mice in each group in each phase were similar at the start of treatment and all mice were weighed weekly during the study. The results showed that the IGFBP-2^{-/-} mice receiving the control peptide gained more weight during the 12 weeks compared to the control wild type mice (FIG. 3A). During the study interval the IGFBP-2^{-/-} mice treated with the HBD1 or HBD2 peptide gained significantly less weight after 8 weeks of treatment compared to IGFBP-2^{-/-} mice treated with the control peptide. This difference persisted in the HBD treated mice compared the control peptide treated mice after 12 weeks of treatment and the difference remained significant (FIG. 3A). In addition, after 10 weeks treatment, the weight gain was significantly less in the mice that received HBD2 peptide, compared to the mice that received HBD1 peptide (FIG. 3A). Importantly, during the study, the gain of body weight in wild type control mice was similar to the IGFBP-2^{-/-} mice treated with either the HBD1 or HBD2 peptides, respectively (FIG. 3A). To exclude the possibility that differences in weight gain were caused by different food intake, food consumption was measured weekly. The results showed no significant difference among all treatments (FIG. 3B). In addition, oral glucose tolerance tests showed that no significant differences were detected in glucose metabolism among different treatments (FIG. 3C).

### The Heparin Binding Domains of IGFBP-2 Inhibit Body Fat Mass Accumulation and Change Serum Adipokine Hormone Concentrations in IGFBP-2^{-/-} Mice.

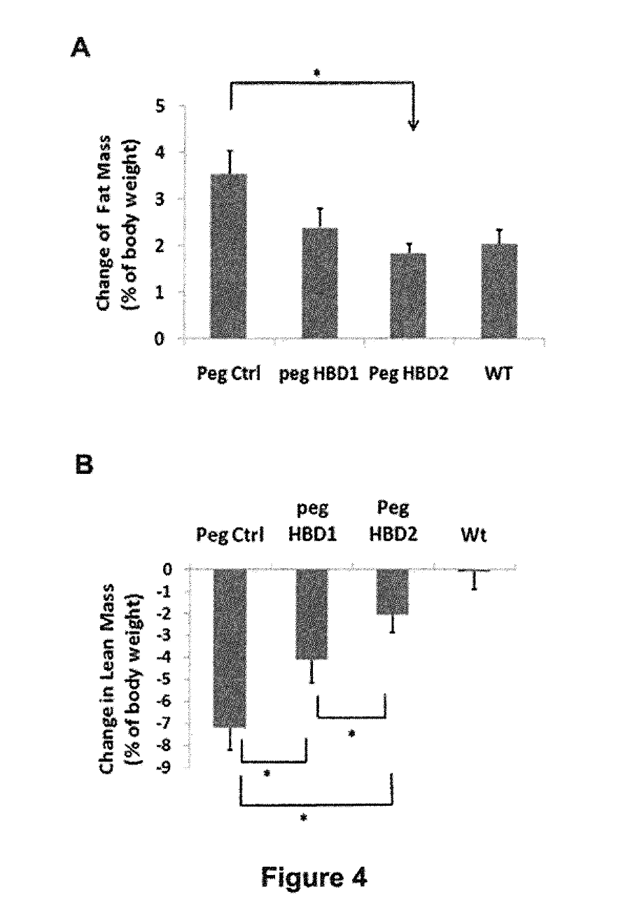

To examine the changes in body composition, MRI was performed on all mice at the beginning and the end of study. The change of fat mass was expressed as change in fat as a percentage of body weight. The results showed that both HBD peptides resulted in a decrease in body fat mass gain over the 12 weeks of the study but only HBD2 treatment reached statistical difference (e.g., 48±9% reduction, p<0.05), compared to control peptide treatment (FIG. 4A). In addition, during the study, IGFBP-2^{-/-} mice treated with a control peptide experienced a severe lean mass loss (e.g., 7.2±1.0% reduction). Both HBD peptides significantly prevented this degree of change but the HBD2 peptide was more potent (e.g., 4.1±1.1% reduction for HBD1, p<0.05; 2.1±0.8% reduction for HBD2) (FIG. 4B).

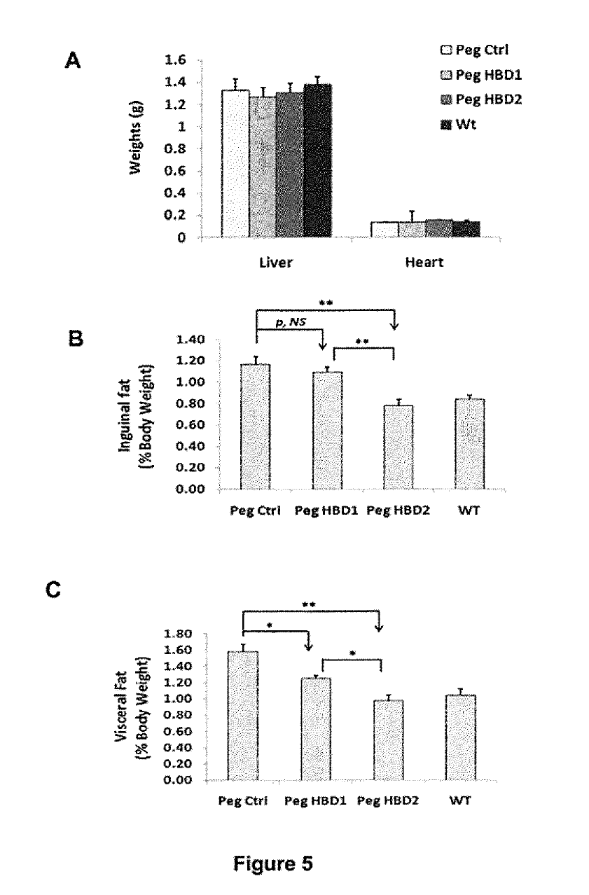

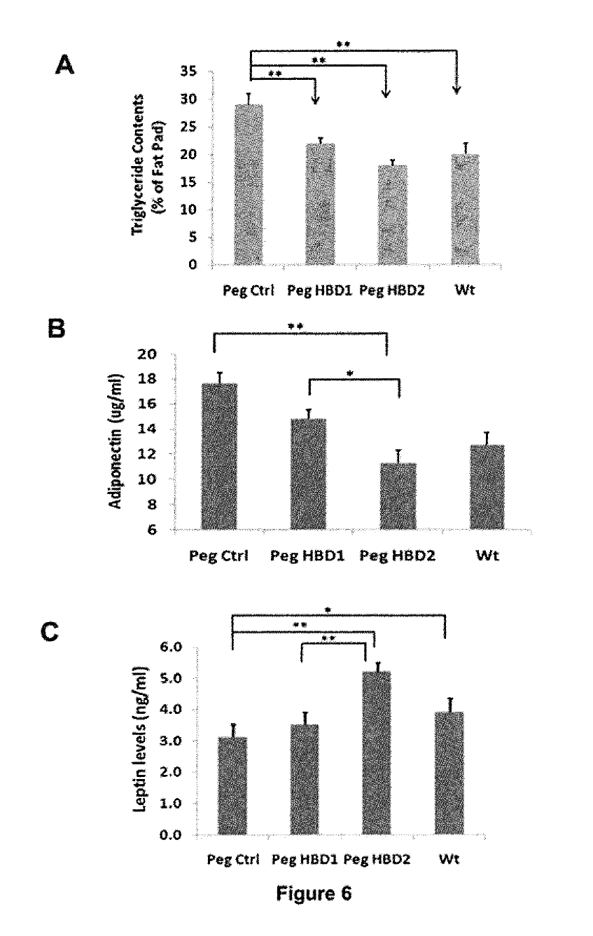

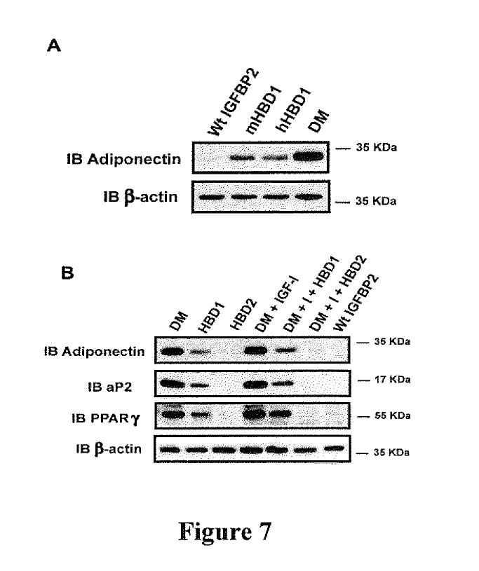

To confirm the effects on fat mass, after completion of 12 weeks of treatment, the mice were sacrificed and two body fat compartments were weighed. The results showed that there was no significant difference in the liver and heart weight among different treatments (FIG. 5A), which excluded the possibility that organ weight change might contribute to the body weight changes that occurred in response to HBD peptide treatment. The fat compartment analysis showed that the IGFBP-2^{-/-} mice given the control peptide had the greatest percentage of inguinal and visceral fat among all groups. The peptide containing the HBD2 sequence significantly inhibited inguinal fat pad weight compared to the IGFBP-2−/− mice treated with the control peptide (e.g., 32±7% reduction, p<0.01) whereas a peptide containing HBD1 sequence had no effect (FIG. 5B). The visceral fat weight was significantly reduced in mice treated with either HBD1 or HBD2 peptide but the peptide containing HBD2 sequence was more effective (e.g., 44±7% vs. 24±5% reduction, p<0.05) (FIG. 5C). Interestingly, when the weights of both fat compartments were expressed as a percentage of body weight the results in IGFBP-2^{-/-} mice treated with. HBD2 peptide were similar to the wild type mice (FIGS. 5B-C). Analysis of the triglyceride content in the inguinal fat pad showed that it was significantly reduced in IGFBP-2^{-/-} mice treated with either HBD1 or HBD2 peptide but HBD2 was more effective (e.g., 37±9% vs. 22±2% reduction, p<0.05) (FIG. 6A). The mean serum adiponectin level was significantly lower in the IGFBP-2^{-/-} mice treated with the HBD2 peptide compared to either the mice treated with the control peptide (e.g., 36±4%, p<0.01) or with HBD1 peptide (e.g., 24±5% reduction, p<0.05) (FIG. 6B). In addition, treatment with the HBD2 peptide significantly increased serum leptin level compared to a control peptide treated mice (e.g., 57±9% increase, p<0.01) whereas the HBD1 peptide had no effect (FIG. 6C). Taken together, these results clearly suggest that both HBD1 and HBD2 peptides are able to inhibit adipogenesis but the HBD2 peptide is a more potent inhibitor.

Comparison of Mouse Peptide KHGRYNLKQCKMSLNGQR with Human Peptide KHGLYNLKQCKMSLNGQR.

Preadipocytes were prepared from mice and grown in culture as described herein. The respective mouse and human peptides were added to cells and the cells were lysed and the lysates were run on SDS gels as described. Immunoblotting for adiponectin was carried as described. Three concentrations of each peptide, 0.7, 2.0 and 6.0 μg/ml, were compared. The results were expressed as % suppression mean±SE(N=3) and were as follows.

| Dose | Mouse | Human | p value |
|---|---|---|---|
| 0.7 | 51.2 ± 8.1 | 49.4 ± 10 | NS |
| 2.0 | 85.6 ± 11.4 | 87.9 ± 6.2 | ″ |
| 6.0 | 95.5 ± 9.9 | 96.9 ± 7.0 | ″ |

These results show that the human and mouse peptide are equipotent in their ability to inhibit adipocyte differentiation.

### Example 2

Insulin-like growth factor binding protein 2 (IGFBP-2) overexpression confers resistance to high fat feeding and inhibits the differentiation of preadipocytes in vitro. However, whether administration of IGFBP-2 can regulate adipogenesis in vivo and the domains that mediate this response have not been defined. IGFBP-2 contains two heparin binding domains (HBD), which are localized in the linker region (HBD1) and C-terminal region (HBD2) of IGFBP2. To determine the relative importance of these domains, synthetic peptides as well as mutagenesis were employed in the studies described herein. Both HBD1 and HBD2 peptides inhibited preadipocyte differentiation but the HBD2 peptide was more effective. Selective substitution of charged residues in the HBD1 or HBD2 regions attenuated the ability of the full length protein to inhibit cell differentiation, but the HBD2 mutant had the greatest reduction. To determine their activities in vivo, pegylated forms of each peptide were administered to IGFBP-2^{-/-} mice for 12 weeks. MRI scanning showed that only the HBD2 peptide significantly reduced (48±9%, p<0.05) gain in total fat mass. Both inguinal (32±7%, p<0.01) and visceral fat (44±7%, p<0.01) were significantly decreased by HBD2 whereas HBD1 reduced only visceral fat accumulation (24±5%, p<0.05). The HBD2 peptide was the more effective peptide in reducing triglyceride content and serum adiponectin but only the HBD2 peptide increased serum leptin. These findings demonstrate that the HBD2 domain of IGFBP2 is the primary region that accounts for its ability to inhibit adipogenesis and that a peptide encompassing this region has activity that is comparable to native IGFBP-2.

The bioavailability of insulin-like growth factor-I (IGF-I) and -II (IGF-II) is modulated by high-affinity IGF-binding proteins (IGFBPs) that regulate ligand transport and bioavailability. IGFBP-2 is the second most abundant IGFBP in human circulation and it is the principal form of IGFBP secreted by white preadipocytes during adipogenesis. Whether it directly inhibits differentiation of non-immortalized preadipocytes isolated from animals and the specific domains within IGFBP-2 that mediate this effect has not been determined. Members of the IGFBP family exhibit 67%-70% structural homology, however, many of the physiological effects of the individual binding proteins are distinct. The greatest homology among the six forms of IGFBPs is contained in the N- and C-terminal regions. The N-terminal region contains the primary IGF-I binding site, while the C-terminal region facilitates IGF-I binding and accounts for the ability of several members of the family to bind to extracellular matrix. A heparin binding domain (HBD) has been identified in the C-terminal region of IGFBP-2, -3 and -5, whereas a RGD sequence is present in IGFBP-1 and -2. In addition to the C-terminal HBD (referred hereafter as HBD2), IGFBP-2 contains a unique HBD that is located in the linker region (referred hereafter as HBD1) (FIG. 1A). Functional studies have shown that C-terminal HBD within IGFBP-3 and -5 can bind to extracellular matrix proteins, which has been proposed to mediate both IGF dependent and -independent actions, whereas the RGD sequence has been shown to be responsible for IGFBP-1 and IGFBP-2 binding the α5β1 integrin and this mediates cell migration. A synthetic peptide containing the HBD1 sequence stimulated osteoblast proliferation, increased trabecular bone mass and reduced bone resorption in IGFBP2^{-/-} mice. However, the roles of the HBD1 and HBD2 domains in altering adipogenesis have not been determined. Therefore the present study was undertaken to determine if IGFBP-2 could inhibit preadipocyte differentiation and to define the relative importance of the HBD1 and 2 domains in regulating this effect. Similarly an in vivo study was performed to determine if peptides containing these sequences could inhibit fat mass acquisition.

Generation of synthetic peptides and peptides pegylation. The synthetic peptide containing the HBD1 domain of mouse IGFBP-2 (CKHLSLEEPKKLRP), a scrambled HBD1 peptide (CKPLRLSKEEHPLK) (HBD1 control peptide), the HBD2 domain of human IGFBP-2 (CKHGLYNLKQCKMSLNGQR) and the C-terminal HBD of IGFBP-5 (RKGFYKRKQCKPSRGRKR) (HBD2 control peptide) were synthesized by the Protein Chemistry Core Facility at the University of North Carolina at Chapel Hill. HBD1 and 2 peptides that did not contain the N-terminal cysteine were also prepared. Purity and sequence identity were confirmed by mass spectrometry. HBD1, HBD2 and HBD1 control peptides (that each contained the N-terminal cysteine) were pegylated following a procedure described herein.

Generation of pLenti-IGFBP-2 wild type (WT), two HBDs mutants and non-IGF-I binding mutant. The wild-type mouse IGFBP-2 amplified from pCMV-SPORT6 (ATCC, Manassas, Va.) was inserted into the pENTR/D-TOPO vector was used as a template to make the substitution mutants. The two IGFBP-2 mutants incorporated substitutions of amino acids within the HBD1 domain containing the sequence ¹⁸⁸KHLSLEEPKKLR¹⁹⁹ and HBD2 domain of IGFBP-2 containing the sequence ²⁴³KHGLYNLKQCKMSLNGQR²⁶⁰. The substitutions, highlighted in bold, were as follows: AALSLEEPAALA (HBD1 mutant) and AAGLYNAAQCAMSLNGQA (HBD2 mutant), respectively. The QuikChange site-directed mutagenesis kit (Agilent Technologies, Santa Clara, Calif.) was used to incorporate the base changes needed to encode these substitutions. The non-IGF-I binding mutant form of IGFBP-2 was prepared as described previously.

Purification of wild type, non-IGF-I binding mutant and two HBD mutant forms IGFBP-2. The constructs were expressed in CHO-K1 cells. Conditioned medium was collected from confluent CHO-K1 cells expressing WT IGFBP2 or non-IGF-I binding mutant or the HBD1 mutant or HBD2 mutant that had been maintained in serum-free α-MEM for 48 h. The expressed proteins were purified. To determine IGF-I binding capacity of non-IGF-I binding IGFBP-2, an IGF-I binding assay was performed.

Cell culture of primary preadipocytes. Preadipocytes were isolated from epididymal fat pads of IGFBP-2^{-/-} mice. The cultures had the medium changed every 2 days until they reached confluency. Two day post-confluent cells were then exposed to differentiation medium (serum free DMEM containing 0.5 mM IBMX, 1 μM dexamethasone, and 5 μg/mL insulin) and incubated for 2 days. Experimental treatments that were added to this medium included either wild type IGFBP-2 (3 μg/ml), HBD1 peptide (6 μg/ml), HBD1 control peptide (6 μg/ml), HBD2 peptide (6 μg/ml), HBD2 control peptide (6 μg/ml), HBD1 mutant IGFBP2 (3 μg/ml), the HBD2 mutant IGFBP-2 (3 μg/ml) or non-IGF-I binding mutant IGFBP-2 (3 μg/ml). Cultures were maintained for two additional days in the standard medium supplemented with insulin (5 μg/ml). Thereafter, the cultures were kept in the fresh standard medium without insulin for additional two days.

Oil Red O staining. Cells were rinsed with PBS and then fixed with 10% formalin for 30 min. 100% propylene glycol (Poly Scientific, Bay Shore, N.Y.) was added and incubated for 5 min and cultures were incubated for 10 min at 60° C. with Oil Red O (Poly Scientific, Bay Shore, N.Y.) and then with 80% propylene glycol for 5 min. Images were captured using an Olympus IX81 inverted microscope and results were quantified using ImageJ software (NIH, version 1.45S).

Immunoprecipitation and immunoblotting. Differentiated adipocytes were lysed in ice-cold lysis buffer and solubilized proteins were quantified (Thermo Scientific, Rockford, Ill.). Equal protein amounts of lysates were loaded onto a SDS-polyacrylamide gel and the proteins were separated, then transferred to an Immobilon filter and visualized by immunoblotting using 1:1000 for anti-adiponectin (Affinity BioReagents, Golden, Colo.), 1:500 for anti-PPARγ (Cell Signaling Technology Inc., Beverly, Mass.), 1:2000 for anti-aP2 (ProSci Inc., Poway, Calif.) and 1:5000 for the anti-β-actin antibody (Sigma Chemical Company, St. Louis, Mo.). The immune complexes were visualized using enhanced chemiluminescence (Thermo Fischer Scientific, Rockford, Ill.).

Mice. The mice, B6.129-Igfbp2^{tmlJep} (referred to as Igfbp2^{-/-} mice) were backcrossed onto C57BL/6J background for at least 10 generations. Igfbp2^{+/+} mice were C57BL/6J controls. All in vivo and ex vivo experimental studies were performed using male mice: Mice were assigned to one of three treatment groups:1) Peg HBD1 peptide (N=8); 2) Peg HBD2 peptide (N=10); 3) Control peptide (N=18). Igfbp2^{-/-} mice were administered 50 μg of each pegylated peptide in 0.1 mL PBS. Igfbp2^{+/+} mice (N=8) given 0.1 mL of PBS served as controls (WT). All injections were administered IP 3 times weekly from 10-22 weeks of age. All mice were provided with free access to 2018 Teklad global rodent diet (Harlan, Dublin, Va.), containing 18.6% protein, 6.2% fat and 3.5% crude fiber. Food consumption and the weights of the mice were determined weekly.

Body composition and serum adipokine measurement. Body fat and lean mass were measured at weeks 0 and 12 of treatment by magnetic resonance imaging analysis (EchoMRI-100, Echo Medical Systems, Houston, Tex.), using unanesthetized animals. After 12 weeks of treatment, the mice were sacrificed and blood was collected by cardiac puncture and centrifuged at 3500 rpm×15 min. The serum was stored at −20° C. Abdominal inguinal and visceral fat pads were dissected from each animal according to defined anatomical landmarks. Subcutaneous fat between the rib cage and the upper thigh was termed subcutaneous inguinal fat, whereas all fat from the lesser curvature of the stomach to the sigmoid colon was termed visceral fat. Fat depots were blotted dry prior to weighing. The right inguinal fat pad was dissected separately and the triglyceride content of this tissue was determined by a calorimetric analysis (Pointe Scientific, Canton, Mich. Serum adiponectin and leptin were measured by ELISA following manufacturer's instructions (Millipore, Billerica, Mass.).

Glucose tolerance test. For oral glucose tolerance tests, following a 4 hr fast, mice were given 20% glucose (2.5 g/kg body weight) by oral gavage and blood was obtained from the tail vein at baseline, 15, 30, 60, 90 and 120 min (Bayer Contour Glucometer, Tarrytown, N.Y.).

Statistical analysis. All data are expressed as the mean E standard error (SEM). Results were analyzed for statistically significant differences using Student's t-test for the data obtained from in vitro assays or ANOVA followed by Bonferroni multiple comparison post hoc test for the data obtained from in vivo assays. In addition, repeated measures-ANOVA was used where appropriate. Statistical significance was set at p<0.05.

Reagents. Immobilon-P membrane was purchased from Millipore Corp. (Bedford, Mass.). Dulbecco's modified Eagle medium (DMEM), streptomycin, and penicillin were purchased from Gibco (Grand Island, N.Y.). The horseradish peroxidase (HRP)-conjugated mouse anti-rabbit and goat anti-mouse antibodies were purchased from Jackson ImmunoResearch Laboratories (West Grove, Pa.). All other reagents were purchased from Sigma Chemical Company (St. Louis, Mo.) unless otherwise stated.

Peptide Pegylation. HBD1, HBD2 and HBD1 control peptides (that each contained the N-terminal cysteine) were pegylated as follows: 10 mg of peptide was mixed with 380 μg of methoxy PEG maleimide (20000 kDa) (1:3 molar ratio peptide to PEG) (JenKem Biotechnology, Allen, Tex.) in 4.0 ml of 0.05M NaPO₄ (pH 7.0) for 14 hr at 4° C. Cysteine (17 mM) was added to block untreated sites. The mixture was desalted using a Zebra Desalt spin column (Thermo Scientific, Rockford, Ill.) following the manufacturer's instructions. Pegylation was verified by SDS-PAGE analysis with Coomassie staining. When pegylated peptides that contained the N-terminal cysteine were compared to non-pegylated peptides that did not contain the N-terminal cysteine, equal molar concentrations had similar effects on preadipocyte differentiation. To exclude the possibility of species differences, the synthetic mouse HBD1 peptide was compared to the human HBD1 peptide. These peptides were also equivalent in inhibiting preadipocyte differentiation. The human HBD1 sequence is identical in bovine IGFBP-2. The intact mouse HBD2 sequence is contained in the HBD1 mutant protein and its bioactivity is similar to the human HBD2 peptide and bovine wild type protein.

Preparation of viral stocks. Viral stocks were generated with 293FT cells (Invitrogen, Carlsbad, Calif.) for each pLenti-construct. Cells were plated at 5×10⁶per 75-cm² plate the day before transfection in growth medium (DMEM with 10% FBS). On the day of transfection, the culture medium was replaced with 5 ml of Opti-modified Eagle's medium-I (Invitrogen, Carlsbad, Calif.) without antibiotics and serum. DNA-Lipofectamine 2000 complexes for each transfection were prepared according to the manufacturer's protocol (Invitrogen, Carlsbad, Calif.). After 14 hr, the medium was replaced with the growth medium. The virus-containing supernatants were harvested 48-72 hr after transfection and centrifuged at 3000 rpm for 15 min at 4° C. The supernatants were filtered and stored as 1-ml aliquots at −80° C.

Generation of pLenti-IGFBP-2 wild type (WT), two HBDs mutants. To generate the HBD1 mutant construct, the following primer was used: 5′-AAGGGTGCCGCAGCCCTCAGTCTGGAGGAGCCCCGCGGCGTTGGCCCCGCCTCC C-3′. To generate the HBD2 mutant construct, two rounds of PCR were performed. The following primer was used for first round PCR: 5′-AACTGTGACGCGGCTGGCCGGTACAACGCGGCGCAGTGCAAGATG-3′. After sequence confirmation, the first round PCR product was used as a template to perform second round PCR. The following primer was used for second round PCR: 5′-AACGCGGCGCAGTGCGCGATGTCTCTGAACGGACAGGCGGGGGAGTGCTGG-3′. The cDNAs encoding the two mutated forms of IGFBP-2 were transferred from pENTR/D-TOPO vector into pLenti6-V5 DEST vector using the LR Clonase reaction.

Isolation of primary preadipocytes. The epididymal fat pads obtained from IGFBP2−/− mice were weighed and the tissue was digested with an equal volume (W/V) of collagenase type A (Roche Applied Science, Indianapolis, Ind.) in HEPES buffer (0.1M and 1.5% BSA) for 45 min at 37° C. The preadipocytes were separated from the stromal vascular cells by centrifugation at 500×g for 5 min. The pelleted cells were washed with DMEM containing 30 mM glucose (Gibco, Grand Island, N.Y.), passed through a 100 μm mesh filter (BD Biosciences, Bedford, Mass.), and then plated in 6-well plates (Falcon Corp., Franklin Lakes, N.J.) at a density of 1.2-1.5×10⁴ cells/ml in DMEM containing 30 mM glucose supplemented with penicillin (100U/ml), streptomycin (100 μg/ml), and 10% FBS (standard medium).

Adipocyte area measurement. Samples of peri-gonadal adipose tissue were fixed in 10% formalin and embedded in paraffin. Multiple sections were obtained from each sample and stained with hematoxylin and eosin. Digital images of each section were acquired using an Olympus BX61 microscope and cell areas were traced manually for at least 200 cells per section by an investigator blinded to the sample identity, using Image J software (1ASS). Four sections from each adipose depot were analyzed to derive the mean cell area per mouse 6 mice per group).

IGFBP-2 and HBD1 or HBD2 peptides inhibit differentiation of IGFBP-2^{-/-} preadipocytes. To investigate the effects of each of the HBDs on adipogenesis, peptides that contained the HBD1 or HBD2 sequences were incubated with cultures of preadipocytes that had been isolated from IGFBP-2 null mice. Native IGFBP-2 inhibited preadipocyte differentiation into mature adipocytes as indicated by suppression of three differentiation markers adiponection, aP2 and PPARγ (FIG. 1A, lane 8). The HBD1 peptide significantly inhibited adiponectin (e.g., 66 d 10% reduction, p<0.01), aP2 (e.g., 51±2% reduction, p<0.01) and PPARγ expression (e.g., 47±9% reduction, p<0.05) (FIG. 1A, lane 2 vs. lane 1). The control HBD1 peptide had no effect (FIG. 1A, lane 3 vs. lane 1). Interestingly, the HBD2 peptide completely inhibited the expression of adiponectin and aP2, and PPARγ expression was reduced 67±7% (p<0.01) (FIG. 1A lane 4 vs. lane 1). A peptide containing the homologous region of IGFBP-5 did not alter their expression (FIG. 1A lane 5 vs. lane 1). IGFBP-2 mutants in which the charged residues in either the HBD1 or HBD2 domains were changed to neutral residues were tested. The IGFBP-2 mutant containing the altered HBD1 sequence inhibited differentiation marker expression as well as wild type IGFBP-2 (FIG. 1A lane 6 vs, lane 8). In contrast, the IGFBP-2 mutant containing the altered HBD2 residues but an intact HBD1 sequence was significantly less effective in preventing preadipocyte differentiation (FIG. 1A lane 7 vs. lane 6). When the ability to form differentiated adipocytes was determined, similar results were obtained (FIG. 2). In addition, a non-IGF-I binding mutant form of IGFBP-2, which had a >5000-fold reduction in IGF-I binding capacity, was utilized. The results showed that this mutant had no inhibitory effect on the differentiation of preadipocytes (FIG. 1B). These results demonstrate that native IGFBP-2, the HBD1 mutant and the HBD2 peptide were the most potent inhibitors of adipogenesis whereas the HBD1 peptide and HBD2 mutant had less activity. Taken together the results strongly suggest that the inhibitory effect of IGFBP-2 on the differentiation of preadipocyte is mediated primarily via its HBD2 domain and also requires the presence of its IGF-I binding capacity.

The HBD1 and HBD2 peptides decrease weight gain in IGFBP-2^{-/-} mice. To investigate the effects of IGFBP-2 HBDs on weight gain and fat mass in vivo, the peptides containing each HBD sequence were administered to IGFBP-2^{-/-} mice. Since both control peptides had a similar effect on preadipocyte differentiation in vitro, only the scrambled HBD1 peptide was used as a control. To extend the half life of the peptides and increase their resistance to proteolysis in vivo, all three peptides were pegylated. It has previously been shown that 50 μg of HBD1 peptide administered via intraperitoneal (IP) injection 5 times per week for 3 weeks promoted bone growth in IGFBP-2^{-/-} male mice. The previous protocol was modified and 50 μg of each peptide was injected (IP) 3 times/week for 12 weeks. Age matched wild-type C57BL/6J (Wt) male mice were treated with vehicle and compared to the three groups of mice that received peptide treatments.

As this study required a large number of IGFBP-2^{-/-} mice, the experiment was separated into two phases. In the first phase, HBD1 and control peptide treatments as well as wild type control mice were studied. In the second phase the effects of the HBD2 and control peptides were determined. The weights and ages of the mice in each group in each phase were similar at the start of treatment. The results showed that the IGFBP-2^{-/-} mice receiving the control peptide gained more weight during the 12 weeks compared to the control wild type mice (FIG. 3A). During the study interval, the IGFBP-2^{-/-} mice treated with the HBD1 or HBD2 peptide gained significantly less weight after 8 weeks of treatment compared to mice treated with the control peptide. This difference persisted in the HBD treated mice and after 12 weeks of treatment the differences remained significant (FIG. 3A). In addition, after 10 weeks, weight gain was significantly less in the mice that received HBD2 peptide, compared to the mice that received HBD1 peptide (FIG. 3A). Importantly, the gain of body weight in wild type control mice was similar to the IGFBP-2^{-/-} mice treated with either the HBD1 or HBD2 peptides, respectively (FIG. 3A). To exclude the possibility that differences in weight gain were caused by differences in food intake, food consumption was measured weekly. The results showed no significant difference among all treatments (FIG. 3B). In addition, oral glucose tolerance tests showed no significant differences among different treatments (FIG. 3C).

The heparin binding domains of IGFBP-2 inhibit body fat mass accumulation and change serum adipokine concentrations. To examine the changes in body composition, MRI was performed on all mice at the beginning and the end of study. After 12 weeks of treatment, both HBD peptides reduced the fat mass gain but only HBD2 reached statistical difference compared to control peptide treatment (e.g., 0.58±0.12 g increase vs. 1.19±0.35 g increase, p<0.05) (Table 1). When the change of fat mass was expressed as a change in fat expressed as a percentage of body weight, the same result was obtained. Only the HBD2 peptide treatment resulted in a significant decrease in body fat mass gain over the study interval (e.g., 48±9% reduction, p<0.05, compared to control) (FIG. 4A). HBD2 peptide treatment was also associated with significant preservation of lean mass compared to control peptide (e.g., lean mass increase: 3.10±0.57 g vs. 1.85±0.49 g, p<0.05) (Table 1). When the lean mass change was expressed as a percentage of body weight, the results showed that IGFBP-2^{-/-} mice treated with the control peptide experienced a relatively greater lean mass change (e.g., 7.2±1.0% relative reduction) compared to mice treated with either HBD peptide, although HBD2 peptide was more effective (e.g., 4.1±1.1% relative reduction for HBD1, 2.1±0.8% relative reduction for HBD2, p<0.05) (FIG. 4B).

To confirm the effects on fat mass, after completion of 12 weeks of treatment, the mice were sacrificed and two body fat compartments were weighed. The results showed no significant difference in the liver and heart weight among different treatments (FIG. 5A), which excluded the possibility that organ weight change contributed to the treatment related body weight changes. Fat compartment analysis showed that the IGFBP-2^{-/-} mice given the control peptide had the greatest inguinal and visceral fat content among all groups. The HBD2 peptide significantly reduced the inguinal fat pad weight compared to IGFBP-2^{-/-} mice treated With the control peptide (e.g., 0.23±0.02 g vs. 0.34±0.03 g, p<0.05) whereas the HBD1 peptide had no effect (Table 2). A similar result was obtained when the inguinal fat pad expressed as a percentage of body weight is compared among the treatments (e.g., 32±7% difference with HBD2 treatment when compared to control peptide, p<0.01) (FIG. 5B).

The absolute visceral fat weight was significantly reduced in mice treated with either HBD1 or HBD2 peptide compared with control peptide treatment but the HBD2 peptide was more effective (e.g., 0.25±0.02 g for HBD2 and 0.34±0.01 g for HBD1 vs. 0.46±0.04 g for control, p<0.05) (Table 2). Similar results were detected when the changes were expressed as a percentage of body weight (e.g., 44±7% difference between HBD2 and 24±5% difference between HBD1 and control, p<0.05) (FIG. 5C). Interestingly, when the weights of both fat compartments were expressed as a percentage of body weight the results in IGFBP-2^{-/-} mice treated with HBD2 peptide were similar to the wild type mice (FIGS. 5B-C). When adipocyte sizes were compared among treatments, no significant differences were detected (e.g., 12399±1408, 12406±748, 11967±716 and 12422±831 pixels/cell for control peptide, HBD1, HBD2 and wild type, respectively. p, N.S.). Analysis of the triglyceride content in the inguinal fat pad showed that it was significantly reduced in IGFBP-2^{-/-} mice treated with either HBD1 or HBD2 peptide but HBD2 was more effective (e.g., 37±9% vs. 22±2% reduction, p<0.05) (FIG. 6A). In addition, the triglyceride content in wild type mice was significantly less than that of IGFBP-2^{-/-} mice treated with a control peptide (e.g., a 31±5% less, p<0.01) (FIG. 6A). The mean serum adiponectin level was significantly lower with the HBD2 peptide treatment compared to either the mice treated with the control peptide (e.g., 36±4%, p<0.01) or with HBD1 peptide (e.g., 24±5% reduction, p<0.05) (FIG. 6B). Treatment with the HBD2 peptide significantly increased serum leptin level compared to control peptide treated mice (e.g., 57±9% increase, p<0.01) whereas the HBD1 peptide had no effect (FIG. 6C). The serum leptin level in wild type mice was also higher than that of control peptide treated IGFBP-2^{-/-} mice (FIG. 6C). Taken together, these results demonstrate that both HBD1 and HBD2 peptides are able to inhibit adipogenesis but the HBD2 peptide is a more potent inhibitor.

As described above, to determine if IGFBP-2 could alter adipogenesis in vivo, synthetic peptides were used. IGFBP-2 has a complex disulfide bonding pattern therefore expression in mammalian cells is required and obtaining sufficient material to be able to treat these animals for 12 weeks would be difficult. Therefore studies were conducted to determine if synthetic peptides that contained sequences derived from two HBDs retained the ability to inhibit preadipocyte differentiation.

The studies reported herein demonstrate that mutagenesis of charged residues in the HBD1 region resulted in loss of the ability of IGFBP-2 to inhibit preadipocyte differentiation in vitro and a peptide containing this region resulted in 31% inhibition of fat mass acquisition. In contrast a peptide containing the sequence within the C-terminal heparin binding domain was a more potent inhibitor of preadipocyte differentiation and an IGFBP-2 mutant that had the charged residues in that region substituted with alanine had reduced ability to inhibit differentiation. The HBD2 peptide was 1.8 fold more potent than the HBD1 peptide in inhibiting preadipocyte differentiation.

These findings were confirmed in in vivo studies in IGFBP-2^{-/-} mice. Administration of the HBD2 peptide significantly inhibited the gain of fat mass over a 12 week period compared to mice that had received a control peptide. Although the HBD1 peptide also had a significant effect, the response to the HBD2 peptide was significantly greater. It was concluded that the HBD2 peptide retains its superior potency in vivo and that both peptides can be utilized as a surrogate for native IGFBP-2. The treatments had no significant effect on food intake but the study was not powered adequately to detect the small difference that was observed (e.g., 8%). The peptide might increase energy expenditure but this was not measured. The changes in fat mass over this interval did not result in a change in glucose tolerance.

To exclude the possibility that HBD peptides inhibit preadipocyte differentiation by antagonizing IGF-1 actions, experiments were conducted wherein IGF-I was added in the presence of each peptide in the differentiation medium. Since the differentiation medium contains 10⁻⁶ M insulin, which is sufficient to activate IGF-I receptor, adding IGF-I had no additional effect on cell differentiation and therefore, no additional effect of HBD peptides could be directly detected. Since IGFBP-2 or HBD peptides cannot bind insulin, it was concluded that IGFBP-2 or the HBD peptides do not inhibit preadipocyte differentiation by preventing IGF-1 or insulin binding to this receptor.

In summary, the studies described herein demonstrate that both heparin binding domains of IGFBP-2 have some ability to inhibit preadipocyte differentiation, however the C-terminal HBD is significantly more potent in vitro and in vivo. The effects of this domain were such that administration of the peptide for 12 weeks limited fat mass accumulation to a rate that was similar to the rate that was measured in wild type mice. This suggests that this domain can mimic many of the effects of the whole protein in limiting adipogenesis. Since IGFBP-2 levels correlate with the development of adiposity and insulin resistance in humans, these findings indicate that this peptide might not only be useful as a pharmacologic tool to study the role of adiposity in mediating insulin resistance but as a potential treatment modality.

### Example 3

Experiments were conducted in which the HBD-2 peptide was administered to genetically normal, ovariectomized mice to determine if it would induce fat loss. The purpose of this experiment was to determine if the peptide could alter fat mass in ovariectomized mice. Estrogen deficient mice are known to gain fat mass following removal of the ovaries. Since the peptide had not been tested in an animal model that had not been genetically manipulated and since it had not been tested in a condition wherein fat mass had been acquired previously to determine if it could reduce pre-existing fat mass this experiment was undertaken. The experimental design was as follows.

There were 8 mice in each treatment group. C57B6 normal mice underwent ovaryectomy at age 8 weeks. They were then maintained on a normal Chow diet (6% fat) for three weeks. At that time they were treated with 2.5 mg/kg of the active peptide injected subcutaneously two days per week for four weeks. The peptide is the pegylated HBD-2 peptide described herein,

A second treatment group that had also undergone ovariectomy was treated with the identical concentration of a control peptide that contained the same amino acids but in a scrambled order. A third group underwent sham surgery wherein an incision was made and then closed and the ovaries were not removed. For comparison the response of mice that had had the IGF BP-2 gene deleted were treated in the same manner.

Eight mice underwent and were treated with the active peptide for four weeks as described for the normal animals. Eight additional mice received the control peptide and eight mice received the sham procedure. The animals were weighed weekly. After 4 weeks they underwent MRI analysis to determine body composition. Both lean mass and fat mass were quantified and the percent fat as a percent total body weight was determined mathematically.

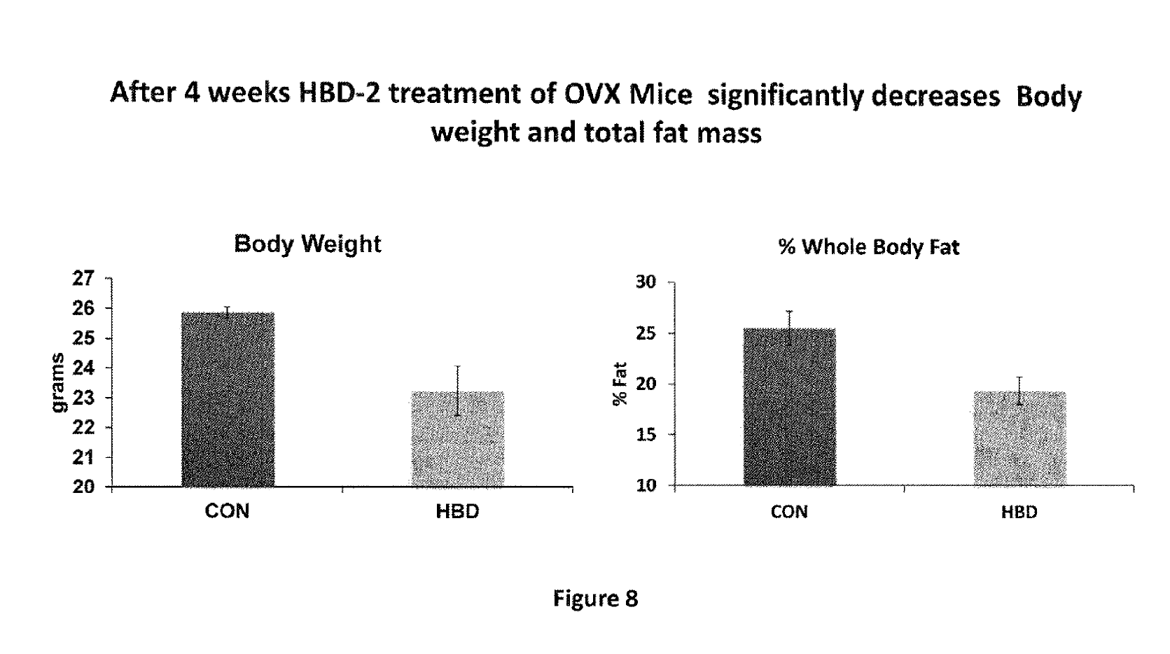

The results demonstrated that the peptide reduced fat mass in the control, genetically normal animals that had undergone ovariectomy. The ovariectomized animals had a highly significant gain in fat mass from 2.83 g in the sham surgery animals to 6.3 g, p<0.001. Administration of the active peptide reduced fat mass from 6.32 g to 4.0, p<0.007. These data are shown in Table 3. Total body weight was also reduced significantly from 25.87 g to 23.0 g p<0.01. This compares to 22.8 g in the sham animals. The animals that underwent ovariectomy that had had the IGF BP-2 gene deleted showed similar changes. Specifically the animals treated with control peptide had 5.37 g of fat whereas the sham animals had 2.9 g.p<0.035. Administration of the HBD-2 peptide resulted in a fat mass of 3.85 g p<0.001 compared to control peptide treated animals. The results comparing the change in fat in the genetically normal animals receiving the active peptide as compared to the control peptide are shown in FIG. 8. In summary the results demonstrate that administration of the HBD-2 peptide to genetically normal animals that have gained fat mass as a result of loss of estrogen causes a significant reduction in fat mass. This indicates that it is likely to be effective in normal individuals who have excessive fat accumulation.

The disclosures of all patents, patent publications and non-patent documents cited herein are incorporated herein by reference in their entirety.

TABLE 1
Fat mass and lean mass increase after treatments

| Treatments | Fat mass increase (g) | Lean mass increase (g) |
|---|---|---|
| Peg Ctrl | 1.19 ± 0.35 | 1.85 ± 0.49 |
| Peg HBD1 | 0.82 ± 0.17 | 2.51 ± 0.35 |
| Peg HBD2 | 0.58 ± 0.12* | 3.10 ± 0.57* |
| Wt | 0.71 ± 0.15 | 2.21 ± 0.40 |

*p < 0.05 when peg HBD2 treatment is compared to peg control peptide (peg Ctrl) treatment.
Data were expressed as mean ± SE.

TABLE 2
Changes in inguinal and visceral fat content after treatments

| Treatments | Inguinal fat (g) | Visceral fat (g) |
|---|---|---|
| Peg Ctrl | 0.34 ± 0.03 | 0.46 ± 0.04 |
| Peg HBD1 | 0.30 ± 0.02 | 0.34 ± 0.01 |
| Peg HBD2 | 0.23 ± 0.02* | 0.25 ± 0.02* |
| Wt | 0.25 ± 0.01 | 0.31 ± 0.08 |

*p < 0.05 when treatment is compared to peg control peptide (peg Ctrl) treatment.
Data were expressed as mean ± SE.

TABLE 3
Body Composition MRI Analysis

| IGFBP-2 | | Body | | | | |
|---|---|---|---|---|---|---|
| Genotype | Treatment | weight (g) | Fat (g) | Total (g) | Fat % | Lean (g) |
| −/− | HBD(pept) | 21.95 ± 0.8 | 3.85 ± 0.4 | 20.90 ± 0.8 | 18.75 ± 1.0 | 16.97 ± 0.4 |
| −/− | Con(pept) | 23.45 ± 0.7 | 5.4 ± 0.4 | 22.85 ± 0.8 | 23.35 ± 1.0 | 17.42 ± 0.5 |
| −/− | sham | 20.86 ± 0.5 | 2.9 ± 0.2 | 19.74 ± 0.5 | 14.84 ± 0.7 | 16.82 ± 0.5 |
| +/+ | HBD | 23.0 ± 0.8 | 4.0 ± 0.4 | 21.65 ± 0.6 | 19.42 ± 1.1 | 17.40 ± 0.3 |
| +/+ | Con | 25.9 ± 0.2 | 6.30 ± 0.5 | 24.57 ± 0.4 | 25.54 ± 1.2 | 18.30 ± 0.3 |
| +/+ | sham | 22.8 ± 0.8 | 2.8 ± 0.4 | 19.73 ± 0.8 | 19.22 ± 1.3 | 16.90 ± 0.6 |

p Values for Comparisons

| | | | | | | |
|---|---|---|---|---|---|---|
| −/− | HBD vs. | 0.05 | 0.035 | 0.011 | 0.018 | 0.385 |
| | Con | | | | | |
| −/− | BD vs. | 0.291 | 0.049 | 0.254 | 0.020 | 0.822 |
| | Sham | | | | | |
| −/− | Con vs. | 0.005 | 0.001 | 0.009 | 0.001 | 0.272 |
| | Sham | | | | | |
| +/+ | HBD vs. | 0.007 | 0.007 | 0.001 | 0.018 | 0.072 |
| | Con | | | | | |
| +/+ | HBD vs. | 0.094 | 0.028 | 0.067 | 0.320 | 0.429 |
| | Sham | | | | | |
| +/+ | Con vs. | 0.010 | 0.001 | 0.001 | 0.001 | 0.037 |
| | Sham | | | | | |

The treatment groups are HBD peptide (HBD pept), Control peptide (control pept) and sham. The sham animals did not have their ovaries removed whereas the other two groups did. The mice have two different genotypes. +/+ are control mice and −/− mice are IGFBP-2 knockout mice.

1. An isolated peptide comprising the amino acid sequence of X₁ X₂ X₃ X₄ X₅ X₆ X₇ X₈ X₉ X₁₀ X₁₁ X₁₂ X₁₃ X₁₄ X₁₅ X₁₆ X₁₇ X₁₈,
wherein:
X₁ is K, H or R;
X₂ is H, R or K;
X₃ is G, A or P;
X₄ is L, R, I or V;
X₅ is Y, F or M;
X₆ is N or Q;
X₇ is L, V or I;
X₈ is K, R or H;
X₉ is Q, N or S;
X₁₀ is C;
X₁₁ is K, H or R;
X₁₂ is M, F, W or Y;
X₁₃ is S, T, N or Q;
X₁₄ is L, V or I;
X₁₅ is N, Q or S;
X₁₆ is G, A, S or P;
X₁₇ is Q, N, S or T; and
X₁₈ is R, K or H.

2. An isolated peptide comprising the amino acid sequence of X₁ X₂ X₃ X₄ X₅ X₆ X₇ X₈ X₉ X₁₀ X₁₁ X₁₂ X₁₃,
wherein:
X₁ is K, H or R;
X₂ is H, R or K;
X₃ is G, A or P;
X₄ is L, R, I or V;
X₅ is Y, F or M;
X₆ is N or Q;
X₇ is L, V or I;
X₈ is K, R or H;
X₉ is Q, N or S;
X₁₀ is C;
X₁₁ is K, H or R;
X₁₂ is M, F, W or Y; and
X₁₃ is S, T, N or Q.

3. An isolated peptide (a) having the amino acid sequence KHGLYNLKQCKMSLNGQR; a peptide (b) having the amino acid sequence of KHGLYNLKQCKMSLNGQR, wherein the K at position 1 is substituted with R or H, the H at position 2 is substituted with R or K, the K at position 8 is substituted with R or H, the K at position 11 is substituted with R or H, the R at position 18 is substituted with K or H, or any combination thereof or c) a pharmaceutically acceptable salt of any of (a) or (b) above, wherein the peptide is not a full length insulin-like growth factor binding protein 2 (IGFBP-2).

4. The isolated peptide of claim 1, wherein the peptide further comprises a polyalkylene glycol moiety coupled to the N terminus thereof, the C terminus thereof, or both the N terminus and C terminus thereof.

5. The peptide of claim 4, wherein the polyalkylene glycol is polyethylene glycol (PEG).

6. The peptide of claim 5, wherein the PEG has a molecular weight from about 10,000 g/mol to about 30,000 g/mol.

7. The isolated peptide of claim 1, wherein the peptide is cyclized.

8. A composition comprising the isolated peptide of claim 1 in a pharmaceutically acceptable carrier.

9. The composition of claim 8, further comprising a weight control agent.

10. A method of inhibiting fat cell differentiation in a subject, comprising administering to the subject an effective amount of the peptide of claim 1.

11. A method of inhibiting weight gain in a subject, comprising administering to the subject an effective amount of the peptide of claim 1.

12. A method of reducing fat mass in a subject, comprising administering to the subject an effective amount of the peptide of claim 1.

13. A method of treating obesity in a subject, comprising administering to the subject an effective amount of the peptide of claim 1.

14. A method of reducing weight in a subject, comprising administering to the subject an effective amount of the peptide of claim 1.

15. A method of controlling body weight in a subject, comprising administering to the subject an effective amount of the peptide of claim 1.

16. The method of claim 15, wherein said amount is effective to reduce weight gain or induce weight loss in said subject.

17. The method of claim 15, wherein said amount is effective in reducing the body mass index of said subject.

18. The method of claim 17, wherein said subject has a body mass index of at least about 25 kg/m².

19. The method of claim 17, wherein said subject has a body mass index of at least about 30 kg/m².

20. The method of claim 15, wherein said administering is continued for a period of at least about 16 weeks or about 24 weeks.

21. The method of claim 15, wherein said administering is continued until said subject has achieved at least 5% weight loss or said subject's body mass index is reduced to less than about 25 kg/m².

22. A method of inducing fat loss in a subject, comprising administering to the subject and effective amount of the peptide of claim 1.

23. The method of claim 10, wherein the subject has insulin resistance.

24. The method of claim 10, wherein the subject does not have insulin resistance.

25.-26. (canceled)
